(19) United States
(12) Patent Application Publication
(10) Pub. No.: US 2019/0239795 A1
(43) Pub. Date: Aug. 8, 2019

(54) EMOTION ESTIMATION APPARATUS, METHOD, AND PROGRAM

(71) Applicant: OMRON Corporation, kyoto-shi, KYOTO (JP)

(72) Inventors: Yasuyo KOTAKE, Kyoto-shi (JP); Hiroshi NAKAJIMA, Kyoto-shi (JP)

(73) Assignee: OMRON Corporation, Kyoto-shi, KYOTO (JP)

(21) Appl. No.: 16/341,958

(22) PCT Filed: Dec. 27, 2017
(86) PCT No.: PCT/IB2017/058414

§ 371 Date: Apr. 15, 2019

(30) Foreign Application Priority Data
Dec. 27, 2016 (JP) 2016-252368
Sep. 1, 2017 (IB) PCT/IB2017/055272

(51) Int. Cl. A61B 5/18; A61B 5/16; A61B 5/0205; A61B 5/024; B60W 40/08

(52) U.S. Cl. CPC A61B 5/18; A61B 5/165; A61B 5/0205; B60W 2040/0818; B60W 40/08; A61B 2503/22; B60W 2040/0872; A61B 5/024

(57) ABSTRACT

A method for assisted driving, includes: storing information indicating an emotion of a subject, and information indicating an activity of the subject; generating learning data representing a relationship between the stored information indicating the emotion of the subject, and the stored information indicating the activity of the subject t and store the learning data into a memory; estimating, after the learning data is generated, a current emotion of the subject based on information indicating a current activity of the subject obtained by an obtaining unit, and the learning data stored in the memory; and providing driving assistance of the vehicle based on the estimated current emotion. Similarly, manufacturing line control, and health care support apparatus are provided, and the apparatuses provide for manufacturing line control and healthcare support on the basis of the estimated emotion.

### FIELD

The present invention relates to an emotion estimation apparatus that estimates the emotions of a subject, a method, and a program.

### BACKGROUND

Techniques for estimating the mental state of humans have been developed to promote safe driving. For example, Patent Literature 1 describes a technique for measuring the vital signs of a subject, such as the heart rate and the blood pressure, obtaining a reaction of the subject to an external event that may influence the subject's mental state, determining an influence on a change in the subject's mental state based on the cognitive reaction, and estimating the subject's mental state based on the influence and the vital signs.

### CITATION LIST

### Patent Literature

Patent Literature 1: Japanese Patent No. 4748084

### SUMMARY

### Technical Problem

To estimate a subject's mental state with the technique described in Patent Literature 1, an external event that may influence the subject's mental state needs to be detected, and a cognitive reaction of the subject needs to be detected at the same time. When, for example, this technique is used for emotion estimation during driving, the estimation apparatus needs to detect traffic congestion, another vehicle cutting into the lane, and other traffic conditions with a vehicle information and communication system VICS (registered trademark) information receiver, a vehicle speed sensor, a radar sensor for a following distance, a camera, or other devices. The apparatus further needs to sense a cognitive reaction of the subject with a sensor, such as a vital sign sensor, at the same time. The estimation apparatus using the above technique thus needs to include an external event monitoring system. This limits the applications of the apparatus, and also cannot avoid a large and complicated design of the apparatus.

One or more aspects of the invention are directed to a simple and widely applicable emotion estimation apparatus that estimates the emotions of a subject without using information about external events, and to a method and a program. More in particular, one or more aspects are directed to an apparatus that can better assist in driving a vehicle, controlling a manufacturing line, or supporting healthcare of a person based on an improved estimation of a mental state of the person, wherein the latter can be easily and accurately obtained by estimating the person's emotion.

### Solution to Problem

According to certain general aspects of the present invention, it is provided an apparatus for estimating a current emotion of a subject based on stored (e.g. previously generated) learning data and on the current activity of the subject; the estimated current emotion can then be used to improve the interaction of the subject with a machine. For instance, in the case of driving a vehicle, a driving assistance can be provided based on the accurately estimated emotion, e.g. the driving assistance can be provided when actually needed in correspondence of a certain estimated emotion; as a result, safer driving can be achieved. In the case of a manufacturing line, a control of the line can be more accurately provided in correspondence of the accurately estimated emotion, such that productivity and/or safety of operation can be achieved. In the case for instance of a healthcare support apparatus, a feedback supporting healthcare can be more accurately provided in correspondence of a more accurately estimated emotion.

A first aspect of the present invention provides an apparatus that obtains information indicating an emotion of a subject, obtains information indicating an activity of the subject, and generates learning data representing a relationship between the obtained information indicating the emotion of the subject and the obtained information indicating the activity of the subject and stores the learning data into a memory. In this state, the apparatus estimates a current emotion of the subject based on the obtained information indicating the current activity of the subject, and the learning data stored in the memory.

A second aspect of the present invention provides an apparatus that generates, when generating the learning data, a regression equation representing a relationship between the information indicating the emotion of the subject and the information indicating the activity with a correct value being the obtained information indicating the emotion of the subject, and a variable being the concurrently obtained information indicating the activity of the subject, and stores the generated regression equation into the memory as the learning data.

A third aspect of the present invention provides an apparatus that obtains information about emotional arousal and emotional valence as information indicating the emotion of the subject, and generates a regression equation representing a relationship between the information indicating the emotion of the subject and the information indicating the activity for the emotional arousal and for the emotional valence, and stores each generated regression equation into the memory as the learning data.

A fourth aspect of the present invention provides an apparatus that defines, when generating the learning data, a plurality of windows each having a predetermined unit duration and being arranged at time points chronologically shifted from one another, and generates, for each window, learning data representing a relationship between a change in the information indicating the emotion of the subject and a change in the information indicating the activity of the subject in each window.

A fifth aspect of the present invention provides an apparatus that generates, for every change of a predetermined value in at least one of the unit duration of the window or the chronological shift of the window, learning data representing a relationship between a change in the information indicating the emotion of the subject and a change in the information indicating the activity of the subject in the window. The apparatus further calculates, for each generated learning data set, a difference between a change in information about an estimate of the emotion obtained based on the learning data and a change in information about a correct value of the emotion, and selects at least one of the unit duration or the chronological shift of the window that minimizes the difference.

A sixth aspect of the present invention provides an apparatus that obtains measurement information including a measurement result of at least one of heart electrical activity, skin potential activity, eye movement, motion, or an activity amount as information indicating the activity of the subject.

A seventh aspect of the present invention provides an apparatus including a learning data updating unit that compares an emotion value estimated by the emotion estimation unit with a range of correct values for the emotion, and updates the learning data stored in the memory based on a result of the comparison.

Further aspects are herein described, numbered as A1, A2, etc. for convenience:

According to aspect A1, it is provided an apparatus for assisting driving of a vehicle, the apparatus comprising:

a storage unit configured to store information indicating an emotion of a subject, and information indicating an activity of the subject, wherein preferably the information indicating an emotion of a subject includes information relating to physiological parameters obtained by means of at least one first sensor set according to a first configuration (see e.g. later described examples of emotional state sensor(s) set according to emotional state sensor configuration(s)), and wherein preferably the information indicating activity of the subject includes information relating to physiological parameters obtained by means of at least one second sensor set according to a second configuration (see e.g. later described examples of activity sensor(s) set according to activity sensor configuration(s)), wherein preferably the first and second sensors are different from each other and/or preferably the first and second configurations are different from each other;

a learning data generation unit configured to generate learning data representing a relationship between the stored information indicating the emotion of the subject, and the stored information indicating the activity of the subject and store the learning data into a memory;

an emotion estimation unit configured to estimate, after the learning data is generated, a current emotion of the subject based on information indicating a current activity of the subject obtained by an obtaining unit, and the learning data stored in the memory; and

an assisting unit configured to provide driving assistance of the vehicle based on the estimated current emotion, wherein the driving assistance preferably includes an active control of the vehicle by the assisting unit during driving.

Preferably, the storage unit is configured to store the information indicating an emotion and the information indicating an activity for a plurality of subjects, each of such information preferably obtained by a respective sensor for a respective subject. Further preferably, the learning data generation unit is configured to generate learning data representing a relationship between information indicating the emotion and information indicating the activity based on the stored information for the plurality of subjects, and further preferably the emotion estimation unit is configured to estimate a current emotion of one subject based on information indicating a current activity of said one subject.

A2. The apparatus according to aspect A1, comprising

a cognitive state estimating unit configured to determine a cognitive state of the subject based on further information indicating an activity of the subject, wherein

the assisting unit is further configured to provide driving assistance of the vehicle further based on the cognitive state.

A3. The apparatus according to aspect A1 or A2, wherein said driving assistance includes any or any combination amongst active control of the vehicle by the assisting unit during driving, and providing a driver of the vehicle with at least a feedback during driving.
A4. The apparatus according to any of aspects A1 to A3, wherein

the information indicating an emotion of a subject is expressed in a two-dimensional coordinate system having a first axis representing an emotional arousal and a second axis representing an emotional valence, and the estimated current emotion is output as values corresponding to the emotional arousal and the emotional valence, and

the assisting unit is configured to provide a degree of driving assistance inversely corresponding to at least one amongst a degree of the emotional arousal and a degree of the emotional valence.
A5. An apparatus for controlling a manufacturing line, the apparatus comprising:

a storage unit configured to store information indicating an emotion of a subject, and information indicating an activity of the subject, wherein preferably the information indicating an emotion of a subject includes information relating to physiological parameters obtained by means of at least one first sensor set according to a first configuration, and wherein preferably the information indicating activity of the subject includes information relating to physiological parameters obtained by means of at least one second sensor set according to a second configuration, wherein preferably the first and second sensors are different from each other and/or preferably the first and second configurations are different from each other;

a learning data generation unit configured to generate learning data representing a relationship between the stored information indicating the emotion of the subject, and the stored information indicating the activity of the subject and store the learning data into a memory;

an emotion estimation unit configured to estimate, after the learning data is generated, a current emotion of the subject based on information indicating a current activity of the subject obtained by an obtaining unit, and the learning data stored in the memory; and

a control unit configured to control the manufacturing line based on the estimated current emotion.

Preferably, the control unit is configured to control one component of the manufacturing line based on the estimated current emotion.

A6. The apparatus according to aspect A5, comprising

a cognitive state estimating unit configured to determine a cognitive state of the subject based on further information indicating an activity of the subject, wherein

the control unit is further configured to control the manufacturing line further based on the cognitive state.

A7. The apparatus according to aspect A5 or A6, wherein the control unit is further configured to perform any combination amongst controlling the speed of movement of a manufacturing line component and controlling the speed of operation of a manufacturing line component.
A8. The apparatus according to any of aspects A5 to A7, wherein

the information indicating an emotion of a subject is expressed in a two-dimensional coordinate system having a first axis representing an emotional arousal and a second axis representing an emotional valence, and the estimated current emotion is output as values corresponding to the emotional arousal and the emotional valence, and

the control unit is configured to provide a degree of control of the manufacturing line inversely corresponding to at least one amongst a degree of the emotional arousal and a degree of the emotional valence.

A9. An apparatus for healthcare support of a subject, the apparatus comprising:

a storage unit configured to store information indicating an emotion of a subject, and information indicating an activity of the subject, wherein preferably the information indicating an emotion of a subject includes information relating to physiological parameters obtained by means of at least one first sensor set according to a first configuration, and wherein preferably the information indicating activity of the subject includes information relating to physiological parameters obtained by means of at least one second sensor set according to a second configuration, wherein preferably the first and second sensors are different from each other and/or preferably the first and second configurations are different from each other;

a learning data generation unit configured to generate learning data representing a relationship between the stored information indicating the emotion of the subject, and the stored information indicating the activity of the subject and store the learning data into a memory;

an emotion estimation unit configured to estimate, after the learning data is generated, a current emotion of the subject based on information indicating a current activity of the subject obtained by an obtaining unit, and the learning data stored in the memory; and

a control unit configured to provide the subject with a healthcare support feedback based on the estimated current emotion.

A10. The apparatus according to aspect A9, comprising

a cognitive state estimating unit configured to determine a cognitive state of the subject based on further information indicating an activity of the subject, wherein

the control unit is further configured to provide the subject with a healthcare support feedback further based on the cognitive state.

A11. The apparatus according to aspect A9 or A10, wherein the healthcare support feedback comprises any combination amongst healthcare support information and health case support stimulus.

A12. The apparatus according to any of aspects A9 to A11, wherein

the information indicating an emotion of a subject is expressed in a two-dimensional coordinate system having a first axis representing an emotional arousal and a second axis representing an emotional valence, and the estimated current emotion is output as values corresponding to the emotional arousal and the emotional valence, and the control unit is configured to provide a degree of healthcare support feedback corresponding to at least one amongst a degree of the emotional arousal and a degree of the emotional valence.

A13. An emotion estimation apparatus, comprising:

a first obtaining unit configured to obtain information indicating an emotion of a subject;

a second obtaining unit configured to obtain information indicating an activity of the subject, wherein preferably the information indicating an emotion of a subject includes information relating to physiological parameters obtained by means of at least one first sensor set according to a first configuration, and wherein preferably the information indicating activity of the subject includes information relating to physiological parameters obtained by means of at least one second sensor set according to a second configuration, wherein preferably the first and second sensors are different from each other and/or preferably the first and second configurations are different from each other;

a learning data generation unit configured to generate learning data representing a relationship between the information indicating the emotion of the subject obtained by the first obtaining unit, and the information indicating the activity of the subject obtained by the second obtaining unit and store the learning data into a memory; and

an emotion estimation unit configured to estimate, after the learning data is generated, a current emotion of the subject based on information indicating a current activity of the subject obtained by the second obtaining unit, and the learning data stored in the memory, wherein

the information indicating an emotion of a subject is expressed in a two-dimensional coordinate system having a first axis representing an emotional arousal and a second axis representing an emotional valence, and the estimated current emotion is output as values corresponding to the emotional arousal and the emotional valence

A14. The emotion estimation apparatus according to any of aspects A1 to A13, wherein

the learning data generation unit generates a regression equation representing a relationship between the information indicating the emotion of the subject and the information indicating the activity with a correct value being the information indicating the emotion of the subject obtained by the first obtaining unit, and a variable being the information indicating the activity of the subject concurrently obtained by the second obtaining unit, and stores the generated regression equation into the memory as the learning data.

A15. The emotion estimation apparatus according to any of aspects A1 or A14, wherein

the learning data generation unit generates a regression equation representing a relationship between the information indicating the emotion of the subject and the information indicating the activity for the emotional arousal and for the emotional valence, and stores each generated regression equation into the memory as the learning data.

A16. The emotion estimation apparatus according to any of aspects A1 to A15, wherein

the learning data generation unit defines a plurality of windows each having a predetermined unit duration and being arranged at time points chronologically shifted from one another, and generates, for each window, learning data representing a relationship between a change in the information indicating the emotion of the subject and a change in the information indicating the activity of the subject in each window.

A17. The emotion estimation apparatus according to aspect A16, wherein

the learning data generation unit includes
a generator configured to generate, for every change of a predetermined value in at least one of the unit duration of the window or the chronological shift of the window, learning data representing a relationship between a change in the information indicating the emotion of the subject and a change in the information indicating the activity of the subject in the window; and
a selector configured to calculate, for each generated learning data set, a difference between a change in information about an estimate of the emotion obtained based on the learning data and a change in information about a correct value of the emotion obtained by the first obtaining unit, and select at least one of the unit duration or the chronological shift of the window that minimizes the difference.
   A18. The emotion estimation apparatus according to any one of aspects A1 to A17, wherein

the second obtaining unit obtains measurement information including a measurement result of at least one of heart electrical activity, skin potential activity, eye movement, motion, or an activity amount as information indicating the activity of the subject.

A19. The emotion estimation apparatus according to any one of aspects A1 to A18, further comprising:

a learning data updating unit configured to compare an emotion value estimated by the emotion estimation unit with a range of correct values for the emotion, and update the learning data stored in the memory based on a result of the comparison.

A20. An emotion estimation method implemented by an emotion estimation apparatus including a processor and a memory, the method comprising:

obtaining information indicating an emotion of a subject;

obtaining information indicating an activity of the subject, wherein preferably the information indicating an emotion of a subject includes information relating to physiological parameters obtained by means of at least one first sensor set according to a first configuration, and wherein preferably the information indicating activity of the subject includes information relating to physiological parameters obtained by means of at least one second sensor set according to a second configuration, wherein preferably the first and second sensors are different from each other and/or preferably the first and second configurations are different from each other;

generating learning data representing a relationship between the obtained information indicating the emotion of the subject and the obtained information indicating the activity of the subject, and storing the learning data into the memory; and

obtaining, after the learning data is generated, information indicating a current activity of the subject, and estimating a current emotion of the subject based on the obtained information indicating the current activity and the learning data stored in the memory, wherein

the information indicating an emotion of a subject is expressed in a two-dimensional coordinate system having a first axis representing an emotional arousal and a second axis representing an emotional valence, and the estimated current emotion is output as values corresponding to the emotional arousal and the emotional valence

A21. An emotion estimation program enabling a processor to function as each component included in the emotion estimation apparatus according to any one of aspects A1 to A19.
A22. Method for assisting driving of a vehicle, the methods comprising steps of:

storing information indicating an emotion of a subject, and information indicating an activity of the subject;

generating learning data representing a relationship between the stored information indicating the emotion of the subject, and the stored information indicating the activity of the subject t and store the learning data into a memory;

estimating, after the learning data is generated, a current emotion of the subject based on information indicating a current activity of the subject obtained by an obtaining unit, and the learning data stored in the memory; and

providing driving assistance of the vehicle based on the estimated current emotion.

A23. The method according to aspect A22, comprising

determining a cognitive state of the subject based on further information indicating an activity of the subject, wherein

providing driving assistance of the vehicle is further based on the cognitive state.

A24. The method according to aspect A22 or A23, wherein said driving assistance includes any or any combination amongst active control of the vehicle by the assisting unit during driving, and providing a driver of the vehicle with at least a feedback during driving.
A25. Method according to any of aspects A22 to A24, wherein providing driving assistance includes providing a degree of driving assistance inversely corresponding to at least one amongst a degree of estimated arousal and a degree of estimated valence included in the estimated current emotion.
A26. Method for controlling a manufacturing line, the method comprising steps of:

storing information indicating an emotion of a subject, and information indicating an activity of the subject;

generating learning data representing a relationship between the stored information indicating the emotion of the subject, and the stored information indicating the activity of the subject and store the learning data into a memory;

estimating, after the learning data is generated, a current emotion of the subject based on information indicating a current activity of the subject obtained by an obtaining unit, and the learning data stored in the memory; and controlling the manufacturing line based on the estimated current emotion.

A27. Method according to aspect A26, comprising further determining a cognitive state of the subject based on further information indicating an activity of the subject, wherein controlling the manufacturing line is further based on the cognitive state.
A28. The method according to aspect A26 or A27, wherein the step of controlling comprises any combination amongst controlling the speed of movement of a manufacturing line component and controlling the speed of operation of a manufacturing line component.
A29. The method according to any of aspects A26 to A28, wherein the controlling step comprises providing a degree of control of the manufacturing line inversely corresponding to at least one amongst a degree of estimated arousal and a degree of estimated valence included in the estimated current emotion.
A30. A method for healthcare support of a subject, the method comprising:

storing information indicating an emotion of a subject, and information indicating an activity of the subject;

generating learning data representing a relationship between the stored information indicating the emotion of the subject, and the information indicating the activity of the subject obtained by an obtaining unit and store the learning data into a memory;

estimating, after the learning data is generated, a current emotion of the subject based on stored information indicating a current activity of the subject, and the learning data stored in the memory; and

providing the subject with a healthcare support feedback based on the estimated current emotion.

A31. The method according to aspect A30, comprising determining a cognitive state of the subject based on further information indicating an activity of the subject, wherein providing the subject with a healthcare support feedback is further based on the cognitive state.
A32. The method according to aspect A30 or A31, wherein the healthcare support feedback comprises any combination amongst healthcare support information and health case support stimulus.
A33. The method according to any of aspects A30 to A32, wherein the providing step further provides a degree of healthcare support feedback inversely corresponding to at least one amongst a degree of estimated arousal and a degree of estimated valence included in the estimated current emotion.
A34. A computer program comprising instructions which, when executed on a computer, cause the computer to execute the steps of any of methods A22 to A33.
A35. An apparatus according to any of aspects A1 to A20, wherein the information indicating an emotion of a subject is expressed in a two-dimensional coordinate system having a first axis representing an arousal state and a second axis representing a valence state, and the estimated current emotion is output as values corresponding to the arousal state and the valence state.
A36. A method according to any of aspects A22 to A33, wherein the information indicating an emotion of a subject is expressed in a two-dimensional coordinate system having a first axis representing an arousal state and a second axis representing a valence state, and the estimated current emotion is output as values corresponding to the arousal state and the valence state.
A37. An apparatus according to aspect A16, wherein the predetermined unit duration of a window is determined on the basis of at least one amongst

obtained activity information indicating an activity state of the subject, and

an interaction time interval indicating a time length for an interacting operation between a device coupled to the apparatus and the subject.

A38. An apparatus according to aspect A37, wherein the device coupled to the apparatus is one amongst a vehicle, a component of a manufacturing line, and a healthcare feedback providing device, and wherein, respectively, the interaction time interval is a time length for an interacting operation between the subject and the vehicle, a time length for an interacting operation between the subject and the component of the manufacturing line, and a time length for an interacting operation between the subject and the feedback providing device.

### Advantageous Effects

As above anticipated, according to certain general aspects of the present invention, it is provided an apparatus for estimating a current emotion of a subject based on stored (e.g. previously generated) learning data and on the current activity of the subject; the estimated current emotion can then be used to improve the interaction of the subject with a machine. Thus, it is possible to more effectively and safely interact with a machine.

Further, the apparatus according to the first aspect of the present invention first generates learning data based on information indicating the emotion of the subject and information indicating the activity of the subject obtained for the same time period, and stores the learning data into the memory. When information indicating the current activity of the subject is obtained in this state, the apparatus estimates the current emotion of the subject based on information indicating a current activity of the subject and the learning data. More specifically, every time when information indicating the activity of the subject is obtained, the current emotion of the subject is estimated in real time based on the obtained information indicating the activity and the preliminarily generated learning data. Thus, the emotion of the subject can be estimated simply by obtaining information indicating the activity of the subject, without monitoring external events, such as environmental conditions around the subject. This simple structure without any component for monitoring external events has a wide range of applications.

The apparatus according to the second aspect of the present invention generates the regression equation with the correct value being the obtained information indicating the emotion of the subject, and the variable being the concurrently obtained information indicating the activity of the subject, and stores the regression equation as the learning data. The emotion of the subject can be estimated through computation using the regression equation, without storing a large amount of learning data.

The apparatus according to the third aspect of the present invention generates the regression equation representing the relationship between the information indicating the emotion of the subject and the information indicating the activity for the emotional arousal and for the emotional valence. The emotion of the subject can thus be estimated for arousal and for valence. The estimation results of the emotion of the subject are then output as information expressed by the arousal and the valence.

The apparatus according to the fourth aspect of the present invention defines the plurality of windows each having a predetermined unit duration and being arranged at time points chronologically shifted from one another, and generates, for each window, the learning data representing the relationship between a change in the information indicating the emotion of the subject and a change in the information indicating the activity in each window. The emotional changes of the subject can thus be estimated in each time period.

The apparatus according to the fifth aspect of the present invention generates, for every change of a predetermined value in at least one of the unit duration or the chronological shift of the window, the corresponding learning data, and calculates, for each generated learning data set, a difference between a change in information about an estimate of the emotion obtained based on the learning data and information about a correct value of the emotion, and selects at least one of the unit duration or the chronological shift of the window that minimizes the difference. This allows the emotion estimation results to be nearer the correct values. The emotional changes of the subject can thus be estimated more accurately.

The apparatus according to the sixth aspect of the present invention measures at least one of the heart electrical activity, the skin potential activity, the eye movement, the motion, or the activity amount, which are correlated with emotions, as information indicating the activity of the subject, and uses the measurement data in generating the learning data and in estimating the emotions. This allows the emotion of the subject to be estimated in a noninvasive manner. In this case, measuring two or more of the above items at the same time increases the accuracy of the estimation.

The apparatus according to the seventh aspect of the present invention updates the learning data when the estimated emotion value deviates from the range of correct values of the current emotion of the subject. This allows the learning data to be updated in accordance with any chronological changes in the learning data or any changes in the learning data over time, and allows the obtained estimates to be constantly near the correct values accordingly.

The above aspects of this invention enable estimation of the emotions of a subject without using information about external events, and provide a simple and widely applicable emotion estimation apparatus, a method, and a program. Further, the application of the estimated emotion to the interaction with machines, as illustrated in other aspects and embodiments, allows to reach an improved and/or safer interaction with the person, and/or improved health conditions for the person.

### BRIEF DESCRIPTION OF THE DRAWINGS

FIG. 1 is an overview of an emotion estimation system according to an embodiment of the present invention.

FIG. 2 is a functional block diagram showing the structure of an emotion estimation apparatus included in the system shown in FIG. 1.

FIG. 3 is a flowchart showing the learning procedure and its details in the emotion estimation apparatus shown in FIG. 2.

FIG. 4 is a flowchart showing the first half part of the procedure and its details for generating and storing learning data in a learning mode shown in FIG. 3.

FIG. 5 is a flowchart showing the second half part of the procedure and its details for generating and storing the learning data in the learning mode shown in FIG. 3.

FIG. 6 is a flowchart showing the procedure and its details in an emotion estimation mode of the emotion estimation apparatus shown in FIG. 2.

FIG. 7 is a diagram describing the definition of emotion information that is input through an emotion information input device in the system shown in FIG. 1.

FIG. 8 is a diagram showing example input results of emotion information obtained through the emotion information input device in the system shown in FIG. 1.

FIG. 9 is a diagram showing the classification of emotion information that is input through the emotion information input device in the system shown in FIG. 1.

FIG. 10 is a diagram showing variations in emotion information that is input through the emotion information input device in the system shown in FIG. 1.

FIG. 11 illustrates a block diagram of a mental state model that is well suited for technical applications wherein a person interacts with a device/machine.

FIG. 12 shows how cognitive and emotional states can be measured by way of objective and repeatable measurements.

FIG. 13 shows examples of objective and repeatable measurements.

FIG. 14A is a block diagram according to embodiment 1;

FIG. 14B is a block diagram according to a variant of embodiment 1, showing in particular how embodiment 1 can be optionally combined with embodiment 2;

FIG. 14C is a flow chart illustrating the operation of embodiment 1;

FIG. 15A is a block diagram according to embodiment 3;

FIG. 15B is a block diagram according to a variant of embodiment 3, showing in particular how embodiment 1 can be optionally combined with embodiment 2;

FIG. 15C is a flow chart illustrating the operation of embodiment 3;

FIG. 16A is a block diagram according to embodiment 4;

FIG. 16B is a block diagram according to a variant of embodiment 4, showing in particular how embodiment 1 can be optionally combined with embodiment 2;

FIG. 16C is a flow chart illustrating the operation of embodiment 4;

### DETAILED DESCRIPTION

The present invention is based, amongst others, on the recognition that estimating the mental state of a person in industrial applications like for instance promoting safe driving of a vehicle, controlling a manufacturing line, or supporting healthcare of a person by means of healthcare devices, it is preferable using an appropriate model that takes into account different types of states of a person, wherein the states are directly or indirectly measurable by appropriate sensors. Thus, the mental state can be objectively and systematically observed, as well as estimated in view of the intended technical application.

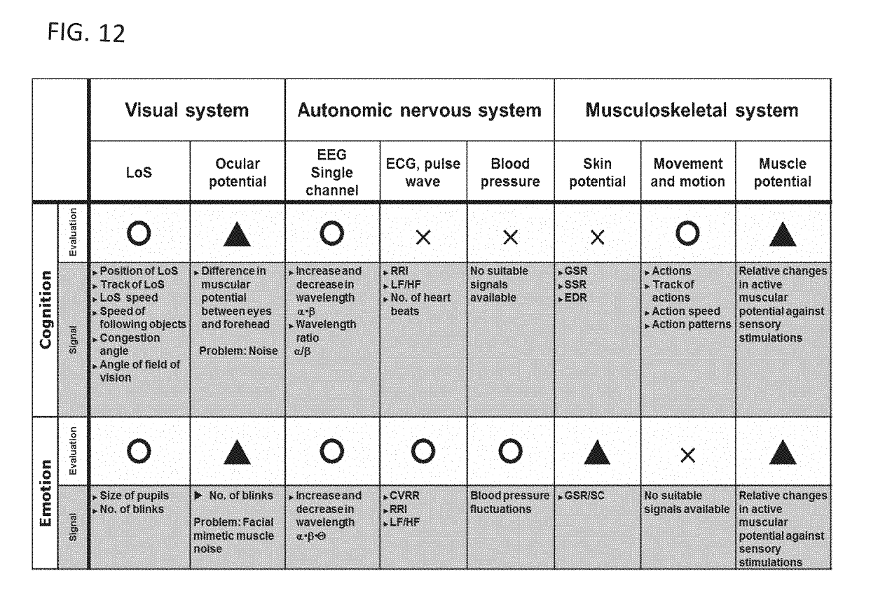

More in detail, a mental state can be modeled by a combination of a cognitive state and an emotional state of a person. The cognitive state of the person relates to, for example, a state indicating a level of ability acquired by a person in performing a certain activity, for instance on the basis of experience (e.g. by practice) and knowledge (e.g. by training). The cognitive state is directly measureable, since it directly relates to the execution of a task by the person. Emotional state has been considered in the past solely as a subjective and psychological state, which could not be established objectively e.g. by technical means like sensors. Other (more recent) studies however led to a revision of such old view, and show in fact that emotional states of a person are presumed to be hard wired and physiologically (i.e. not culturally) distinctive; further, being based also on arousal (i.e. a reaction to a stimuli), emotions can be indirectly obtained from measurements of physiological parameters objectively obtained by means of suitable sensors, as also later mentioned with reference to FIG. 12.

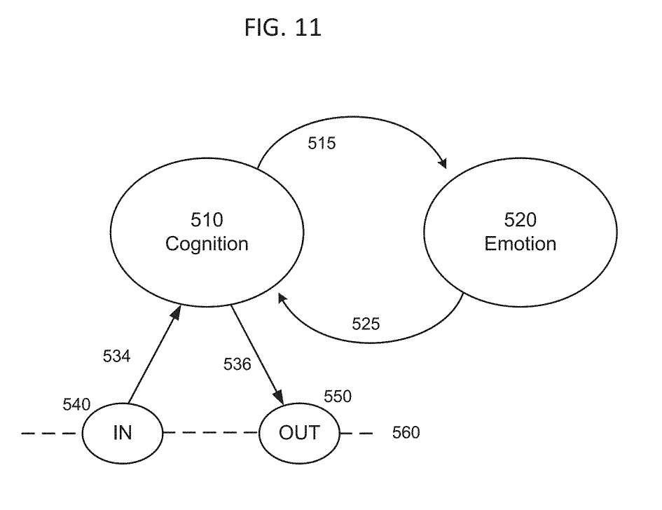

FIG. 11 shows a model of a mental state that can be used, according to the inventors, for technical applications like promoting safe driving (it is noted that the same model can be applied to other applications including controlling a manufacturing line, or supporting healthcare of a person, and more in general to any circumstances where there is an interaction between a person and a device/machine). In particular, the model comprises a cognitive part 510 and an emotional part 520 interacting with each other. The cognitive part and the emotional part represent the set of cognitive states and, respectively, the set of emotional states that a person can have, and/or that can be represented by the model. The cognitive part directly interfaces with the outside world (dashes line 560 represents a separation to the outside worlds) in what the model represents as input 540 and output 550. The input 540 represents any stimuli that can be provided to the person (via the input “coupling port” 540, according to this schematic illustration), and the output 550 (a schematic illustration of an output “coupling port” for measuring physiologic parameters) represents any physiological parameters produced by the person, and as such measurable. The emotional part can be indirectly measured, since the output depends on a specific emotional state at least indirectly via the cognitive state: see e.g. line 525 (and 515) showing interaction between emotion and cognition, and 536 providing output, according to the model of FIG. 11. In other words, an emotional state will be measurable as an output, even if not directly due to the interaction with the cognitive part. It is herein not relevant how the cognitive part and the emotional part interact with each other, and it is in fact referred to respective theories and studies. What matters to the present discussion is that there are input to the person (e.g. one or more stimuli), and output from the person as a result of a combination of a cognitive state and an emotional state, regardless of how these states/parts interact with each other. In other words, the model can be seen as a black box having objectify measurable input and output, wherein the input and output are causally related to the cognitive and emotional states, though the internal mechanism for such causal relationship are herein not relevant.

Despite the non-knowledge of the internal mechanisms of the model, the inventors have noted that such a model can be useful in practical and technical applications in the industry, like for instance when wanting to improve safety in driving vehicles, as it will also become apparent in the following.

FIG. 12 shows how cognitive and emotional states can be measured by way of objective and repeatable measurements, wherein a circle, triangle and cross indicates that the listed measuring methods are respectively well suitable, less suitable (due for instance to inaccuracies), or (at present) considered not suitable. Other techniques are also available, like for instance image recognition for recognizing facial expressions or patterns of facial expressions that are associated to a certain emotional state. In general, cognitive and emotional states can be measured by an appropriate method, wherein certain variable(s) deemed suitable for measuring the given state are determined, and then measured according to a given method by means of suitable sensor(s). As also evident from FIG. 12, the emotional state can be obtained by measuring respective physiological parameters (see e.g. FIG. 12) by at least one emotional state sensor according to an emotional state sensor configuration, and the cognitive state can be measure by at least one cognitive state sensor according to a cognitive state sensor configuration, wherein the at least one emotional state sensor is different from the at least one cognitive state sensor and/or the emotional state sensor configuration is different from the cognitive state sensor configuration. For instance, with reference to FIG. 12, LoS (Line of Sight) measurements can be performed for estimating or determining the cognitive state and/or the emotion state, however the configuration of the sensor is different since the parameter(s)/signal(s) used is different depending on whether the emotion or cognition wants to be determined. An example of the sensor for obtaining LoS is represented by a camera and an image processing unit (either integrated or separated from the camera), wherein the camera and/or the processing unit are differently set in order to acquire a signal related to the cognitive state (e.g. any one or a combination of the following examples: the position of LoS, the track of LoS, the LoS speed, the speed of following objects by the eye(s), the congestion angle, and/or the angle of field of vision, etc.) or a signal related to the emotion state (any one or a combination of the following examples: size of pupils, number of blinks, etc.). For example, if the number of blinks wants to be detected, the camera should be set to acquire a given number of images (or a video with a given, preferably high, number frames per second) and image processing unit for recognizing one blink; when the position of LoS wants to be detected, the camera may be set to acquire just one image, even if more is preferable, and the image processing unit to detect the LoS position from the given image(s). Similar considerations apply to other signals relating to LoS for either cognitive state or emotional state; also, similar considerations apply to other types of signals like those relating to the autonomic nervous system or musculoskeletal system as directly evident from FIG. 12. With this regard, it is also noted that (at least according to the present knowledge) blood pressure measurements are suitable for detecting the emotional state, but not the cognitive state: thus, in this case, any blood pressure sensor would be suitable for obtaining an emotional state, and any sensor suitable for obtaining blood pressure would be an example of the emotional state sensor regardless of its configuration. Similarly, any sensor suitable for detecting movement and motion (e.g. any or a combination of: actions, track of actions, action speed, action patters, etc., see FIG. 12) is an example of a cognitive state sensor regardless of its configuration. Thus, as also shown in FIG. 12, a cognitive state and an emotional state can be detected by a cognitive state sensor and, respectively, emotional state sensor, and/or—when the sensor itself can be the same or similar—by a different configuration of the sensor. Herein, by sensor it is meant a sensing device for detecting physical signals, possibly together (as necessary) with a processing unit for obtaining information on the cognitive or emotion state on the basis of the physical signal. With reference to the emotional state sensors, it is noted that for instance the emotional state can be obtained on the basis of (i) brain related parameter(s) and/or (ii) appearance related parameter(s) and/or other parameter(s).

(i) The brain related parameter(s) obtained by suitable sensors and/or sensor configuration(s) (see also FIG. 12):

The brain related parameter(s) can be represented for example by brain waves obtained by EEG, e.g. by detecting an event-related potential ERP (defined as a stereotyped electrophysiological response to a stimulus). More in particular, using a relationship between the applied stimuli (ex. music, picture for relaxing, excitement, etc.) and the measured EEG pattern corresponding to the ERP induced by a (preliminary learned/known or learned for each user) stimuli, it is possible to determine whether the specific characteristic of the EEG is associated with a known emotional state (e.g. appearances of alpha waves when relaxing). In other words, according to this example, by observing the EEG pattern, and specifically the ERP, it is possible to obtain an indirect measure of the emotional state. For more on ERP, see e.g. An Introduction to the Event-Related Potential Technique, Second Edition, Steven J. Luck, ISBN: 9780262525855.

According to another example, the brain blood flow obtained by fMRI (functional Magnetic Resonance Imaging) can be used as a brain related parameter: the active region of the brain, in fact, can indicate some emotional states; for example, the correlations of BOLD (blood oxygen level dependent) signal with ratings of valence and arousal can be obtained in this way, thus achieving an indirect measure of the emotional state (see e.g. The Neurophysiological Bases of Emotion: An fMRI Study of the Affective Circumplex Using Emotion-Denoting Words, by J. Posner et al, Hum Brain Mapp. 2009 March; 30(3): 883-895, doi: 10.1002/hbm.20553).

The above measurements methods/devices can be also combined together. Techniques based on (i) are accurate, but the measurement device may be large and the user's motions may be largely limited. (ii) Appearance related parameter(s) can be obtained from suitable sensors and/or sensor configurations (see also e.g. FIG. 12), for instance on the basis of:
Facial image analysis of facial expression(s) (as captured for instance by a camera): for instance, using pixel information such as RGB value and intensities, one or more parameters including the angles of the eyebrows, the angle of the mouth, the degree of mouth opening, and/or the degree of eye openings are calculated; the emotion can then be determined (preferably, automatically by a hardware/software unit) based on the combination of one or more such parameters using available set of templates defining the relationship between those parameters and emotions.
Acoustic analysis of voice expressions: similar to the facial expressions, the emotion can be determined using the available set of templates defining the relationship between the parameters and emotions.

A combination of facial expression and voice expressions can also be used. Emotions estimated on the basis of appearance related parameter(s) are estimated with an higher/increased accuracy when the information amount increases, e.g. when the amount of parameters used increases, or (mathematically speaking) when using a higher dimensional information. In simpler words, when acoustic analysis and facial analysis are both executed, and/or when facial analysis is performed on the basis of multiple analysis on eyebrows, angle of mouth, etc., then accuracy can be increased. The more the parameters used in the analysis, however, the larger the computing resources needed for processing; moreover, providing/arranging camera for each user or requesting the voice utterances may not always be possible depending on the situations. Thus, the higher accuracy comes at a price in the terms of computational resources and/or complexity of the camera/machines used for such analysis. (iii) Other parameters, possibly obtained by other sensors and/or different configurations of sensors (see e.g. FIG. 12), can be used for estimating emotions, like for instance:
Pupil size by eye image recognition (i.e. an analysis made on image(s) taken of the eye(s) of a subject), wherein the Time Resolution TR is preferably higher than 200 Hz, for example;
Heart electrical activity, detected by ECG, preferably having TR higher than 500 Hz, for example.

Techniques based on (iii) are accurate, but may require large computing resources in analysis. By any one of or any combination of above techniques, including (i) to (iii), emotional state can be sensed; however, for sensing the emotions accurately, fluctuations of the states, or the continuous variations of the states are important information to consider, which require relatively high time resolution and high dimensional information (thus resulting in high computing resources). Thus, the activity sensor is a sensor that requires smaller information amount, and/or less processing load (including processing time), and/or less time resolution, and/or constructionally simpler and/or less complex than the emotional sensor. As said above, in one example, an activity is an activity/task performed by the subject when interacting with a device.

As anticipated, a variety of sensors are suitable for obtaining such measurements, and are herein not described since any of them is suitable as long as they provide any of the parameters listed in FIG. 12, or any other parameters suitable for estimating cognitive and/or emotional states. The sensors can be wearables, e.g. included in a wrist or chest wearable device or in glasses, an helmet like device for measuring brain activity from the scalp (e.g. EEG/NIRS), or a large machine like PET/fMRI.

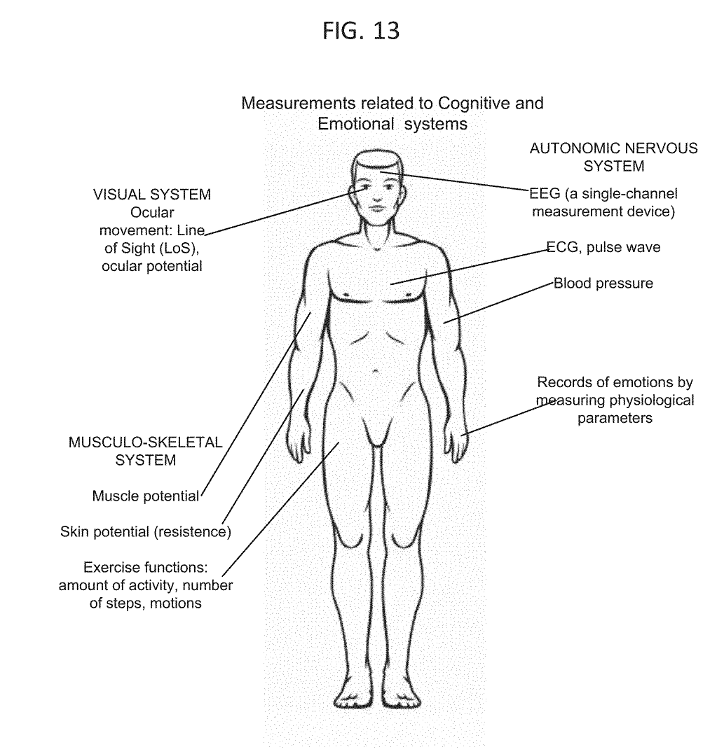

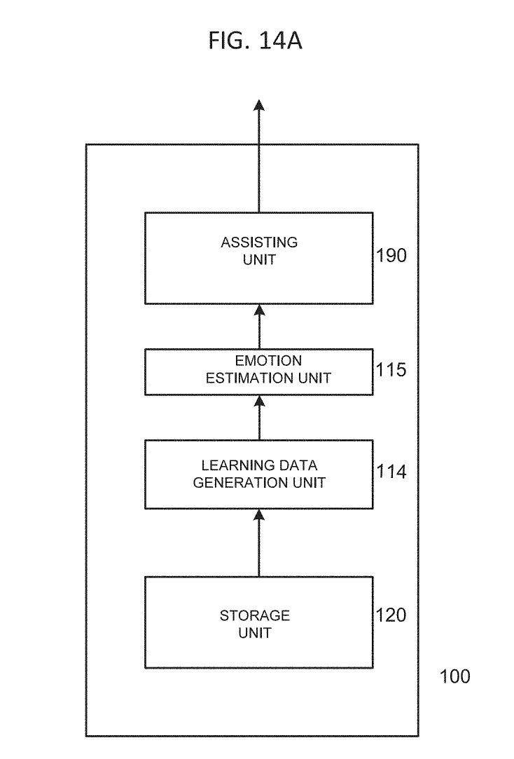

Thus, it possible to model a person, like for instance a driver of a vehicle (or, in other applications, an operator of a factory production line; an operator of a machine; or a person using a device for providing healthcare support), by using a model as illustrated in FIG. 11, and collect measurements of physiological parameters of the person as shown in FIGS. 12 and 13. In this way, as also shown in the following, it is possible for instance to improve safety of driving, or safety/productivity/level of quality of a manufacturing line, or health status of a person.

The above explanation is provided as illustrative and propaedeutic to the understanding of the invention and following embodiments/examples, without any limitation on the same.

Turning to the present invention, and referring for the sake of illustration to the application of promoting safe driving, learning data are generated representing a relationship between information indicative of an emotional state of a subject (i.e. a person) and information indicative of an activity of the person. As herein explained, the information on the emotional state (used to generate the learning data) can be acquired indirectly by means of suitable measurements made on the subject, see also the above discussion in relation to FIGS. 12 and 13, in particular and preferably by means of devices suitable for determining such state with high precision (regardless of the size and complexity of the sensor or device used; preferably, such sensors are large and complex devices achieving higher accuracy than other sensors as those included in wearables). Though not necessary, also a direct indication of such an emotional state (used to generate the learning data) can be obtained, for instance by having a person's emotional state directly input by the person. Also, a combination of both indirect and direct determination of the emotional state is possible.

The learning data can be obtained, as also later explained in more detail, on the basis of information indicating an emotion of at least one subject and information indicating an activity of the same at least one subject, wherein such information have been collected by means of suitable sensors for at least one subject, and preferably stored. The information indicating an emotion of a subject includes information relating to physiological parameters (related to an emotional state of a subject and) obtained by means of at least one emotional state sensor (an at least one first sensor) set according to an emotional state sensor configuration (a first configuration of the corresponding at least one first sensor). Reference is also made to the above discussion in relation to FIG. 12 giving examples of the emotional state sensor and the emotional state sensor configuration. The information indicating activity of the at least one subject includes information relating to physiological parameters (relating to an activity performed by the at least one subject and) obtained by means of at least one activity sensor set according to an activity sensor configuration. The emotional state sensor and the activity sensor are different from each other and/or the emotional state sensor configuration and activity sensor configuration are different from each other.

For instance, as also later described, the activity sensor can be a sensor capable of measuring heart electrical activity H, skin potential activity G, motion BM, activity amount Ex, etc. With reference to the example of heart electrical activity H, the activity sensor (or a suitable configuration of a sensor suitable for measuring heart electrical activity) is capable of measuring the heartbeat interval (R-R interval, or RRI), and/or the high frequency components (HF) and/or the low frequency components (LF) of the power spectrum of the RRI, with a required Time Resolution (TR) preferably set to 100 Hz-200 Hz. Such parameters can be obtained for instanced by means of an ECG device and/or a pulse wave device. As discussed above, see e.g. the other parameters (iii) used for measuring emotions, heart activity can be used also for estimating emotions; however, the sensors used for measuring heart activity related to emotions must be set differently that the same sensors when used for measuring heart activity related to an activity performed by the subject; in the example herein discussed, for instance, a TR of 100-200 Hz suffices for measuring activity, while a TR of 500 Hz or more is preferable for measuring emotions. This means that that activity measurement can be achieved with less computational resources than emotion measurements. Regardless of the complexity necessary for obtaining activity information and emotional information, both are used—once obtained—in order to generate learning data indicating a relationship between activity information and emotional information. The thereby obtained learning data can be stored, and used to estimate a current emotional state of a person. In fact, when information about a current activity of a subject are obtained, the learning data can be used together with the obtained information about a current activity to estimate the emotional state of the subject. The sensors used for estimating the current activity can be any (or any combination) of those described above with reference to FIGS. 12 and 13. Preferably, the sensors need not be as accurate as those used to indirectly acquire the emotional state used for generating the learning data. A wearable sensor may thus suffice, in one example.

By making reference to the example of heart activity, it has been said that the activity information can be more easily (in terms of less computational resources) obtained than emotion information when starting from heart activity measurements; thus, thanks to the obtained learning data, it is possible to obtain the current emotion of a subject on the basis of the measured activity when the system is in operation (i.e. when using the learning data). In other words, it is not necessary to measure the current emotion of the subject when the same performs an activity (like driving a vehicle, working on a factory line, or doing something else when carrying a healthcare device, etc.); the emotion can be derived, thanks to the learning data, from the easier-to-obtain activity information.

The activity information can be obtained, as also later discussed, by other measurements like for instance based on any one or any combination of:
Skin potential activity G, e.g. by measuring the galvanic skin response (GSR); this is a parameter easier to obtain, when compared to parameters used to measure an emotional state;
The eye movement EM, e.g. by measuring the eye movement speed and the pupil size (e.g. based on captured images(s) or video(s) on a subject); in this case, when noting that the same or similar parameters can be used also for obtaining emotions (see (iii) above), the required TR may be equal to or lower than 50 Hz (fluctuations or continuous variations of the sensed parameter is not obtained within this range of TR). Similarly to the case of heart activity, the EM measurements related to the activity of the subject is easier to obtain that the EM measurements related to emotions.

The motion BM, like e.g. the hand movement speed. This is also a parameter that is easier to obtain than parameters related to emotions. In general, therefore, activity information are easier to obtain either because they can be obtained by less complex sensors than those required for measuring emotions, or—when the same type of sensors are used—the configuration of the sensor for acquiring activity information results in less computing resources than the configuration for acquiring emotions. Thus, by using learning data and the (easily) acquired activity information, it is possible to obtain the emotional state of a subject. As a consequence of obtaining the emotional state, safer driving, improved manufacturing, and improved health conditions can be conveniently achieved by easily taking into account the mental state of a subject interacting with a device.

Moreover, the learning process may be performed on activity and emotion data obtained from one single subject. However, it is preferable that the learning is performed on activity and emotion information obtained from a plurality of subjects, such that the relationship can be more accurately learned or found. The learning data (obtained as a result of such learning process) can then be used for estimating the emotion of a subject; such subject can be one of the plurality of subjects to which the activity and emotion information used in the learning process refer, or anther subject not belong to such plurality of subjects.

Further, the activity and emotion information for a plurality of subjects need not be measured in the same way for all subjects of the plurality. In other words, for a first group of subjects (the group including at least one subject), activity and emotion information can be obtained according to a first type of measurements characterized by first emotional state sensor and first activity sensor and/or respective configurations; similarly for a second, third, etc. group of subjects, the respective activity and emotion information can be obtained by a second, third, etc. type of measurements. In this way, it is possible to obtain learning data capable of being used in a variety of situations, and thus leading to overall more accurate results.

In addition, the activity information and emotion information (on which the learning process is then performed) can be obtained for a given subject, preferably when the subject is performing a certain task. Further preferably, the certain task belongs to a set of tasks including at least one task characterized by interaction between the subject and a device. For instance, if the device is a vehicle, the task can be represented by a driving operation of the vehicle (a driving type of task), and the activity and emotion information are obtained when the subject is driving, e.g. by means of sensors and/or sensor configurations compatible with driving. In another example, the task relates to performing an operation on a production line (a manufacturing type of task), and the emotion and activity information are obtained while the subject(s) performs the task in the production line. In another example, the task relates to an action performed when the subject is coupled to a health care device (a healthcare related type of task), and the emotion and activity information are obtained when the user performs such action. The learning process can be performed on data referring to activity and emotion information for one or more subjects performing the same or different types of task.

By estimating the emotional state on the basis of the learning data and the sensed activity, it is possible to use overall less sensors, since no external event needs to be monitored, which is in contrast to prior art techniques discussed in the introduction. In addition, the emotion can be obtained in an easy way, since obtaining the activity information is technically easier than measuring the emotional state. Furthermore, the mental state of the person can be obtained with high accuracy thanks to the fact that the emotional state is obtained: in fact, the estimation of the emotional state allows achieving a more accurate estimation of the overall mental state than when using other techniques aimed at estimating only the cognitive state (i.e. only those parts of the mental state that are directly but not indirectly measurable from the outside, see the discussion in relation to FIG. 11). In this way, applications like increasing drive safety can highly benefit. In fact, based on the emotion estimation (more accurately representing the current mental state), it is for instance possible to provide driving assistance more accurately, and thus increasing safety when driving. For example, in applications like promoting drive safety, automatic systems can automatically react when a potential hazardous situation is detected, wherein the hazardous situation is linked to the detection of a mental state deemed as hazardous. Thus, if the mental state can be more accurately determined, the automatic reaction can more accurately obtained, and an increased safety achieved. In other words, the easy-to-obtain emotion estimation leads to a more accurate mental state estimation, on the basis of which an improved driving assistance can be provided, as also later further details. Similarly, in applications like controlling a manufacturing line, it is possible to better control the manufacturing line on the basis of the emotion estimation of an operator of the same line, such that productivity, safety, and/or level of quality of the manufacturing line can be obtained. Similarly, a device for supporting healthcare of a person can be obtained, wherein the person is provided with a feedback supporting health care on the basis of the estimated emotion, so that the person's health status can be improved or more easily maintained. Moreover, the estimation of the emotional state can be optionally combined with the detection of a cognitive state in order to further increase the accuracy in the estimation of the overall mental state.

Reference has been made to the case of drive safety wherein the driver interacts with the vehicle, or an operator working on a production line, or a person using a device supporting healthcare; however, this is provided only as an example of a person interacting with a machine or device, and in fact what is herein described applies to any type of interaction between a person and the device or between the person and the operation of such device. The device can be for instance an industrial machine or industrial device, a domestic appliance, an office appliance, a vehicle of any type, etc.

Embodiments of the present invention will now be described with reference to the drawings.

### Embodiment 1—Apparatus for Assisting Driving of a Vehicle

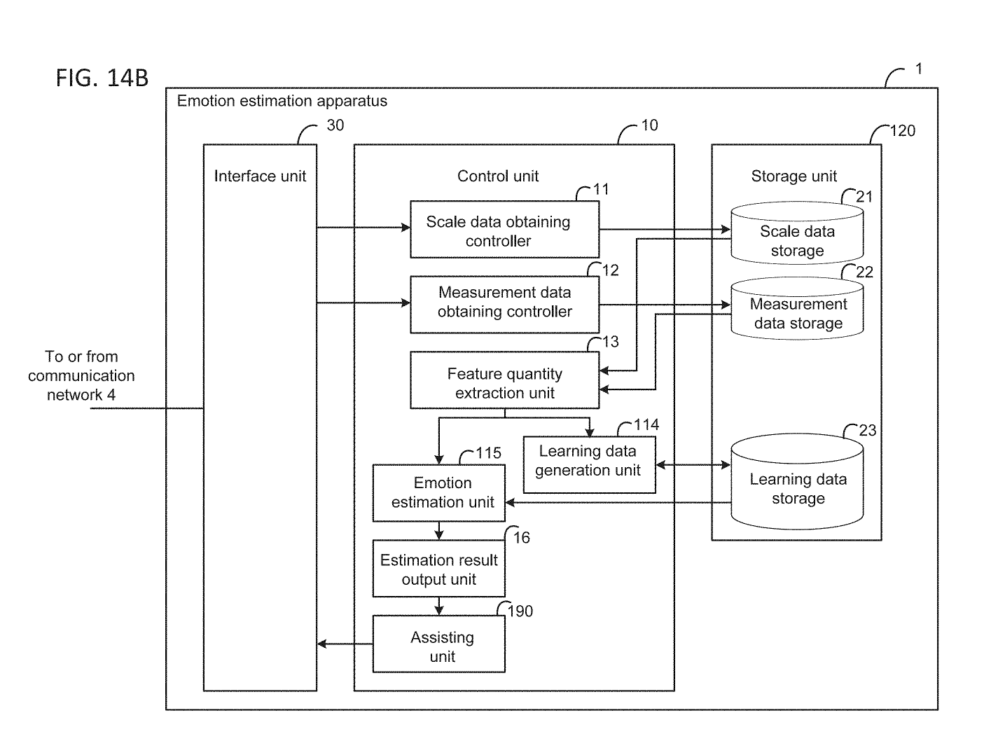

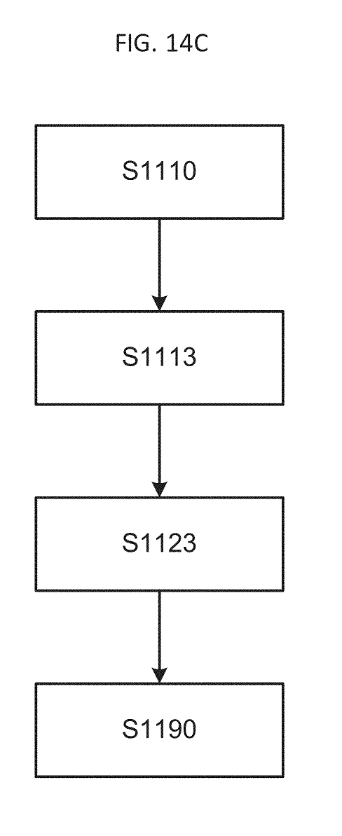

FIG. 14A shows an apparatus 100 for assisting driving of a vehicle, including a storage unit 120, a learning data generation unit 114 and an emotion estimation unit 115, and an assisting unit 190. The storage unit 120 is configured to store information indicating an emotion of a subject, and information indicating an activity of the subject. For instance, and as also explained above, if PET/f MRI is used, the measurement result of PET/fMRI is used to determine the information on emotion of the subject. As explained above, however, the emotion information can be obtained also via less complex or smaller sensors, though this would require large computational resources. At the same time, other parameters can be measured (the same as measurable by wearables), which will be part of the information indicating activity of the subject, as also above discussed. The learning data generation unit 114 generates learning data representing a relationship between the stored information indicating the emotion of the subject (preferably obtained by a first obtaining unit, discussed for instance in embodiment 2), and the stored information indicating the activity of the subject (preferably obtained by a second obtaining unit, also discussed in embodiment 2) and store the learning data into a memory. The emotion estimation unit 115 estimates, after the learning data is generated, a current emotion of the subject based on information indicating a current activity of the subject obtained by an obtaining unit (e.g. the second obtaining unit; the current activity can be obtained or acquired in correspondence of the estimation or of performing the estimation, though not necessarily exactly at the same time), and the learning data stored in the memory. When the sensor used to collect measurement of physiological parameters related to the emotion estimation is a device difficult to wear or to move around (like e.g. a fMRI device), and the data need to be collected for an application like driving or working in a factory where the use of such sensor/device is not easy, then the emotion data can be collected at a first point in time, and the activity information at a second point in time sufficiently close to the first point in time. When instead the emotion and the activity related measurements want to be taken at the same point in time, or sufficiently close points in time, then the emotion can be estimated on the basis of sensors that are more easily wearable, or that can be more easily carried around, or to which the subject can be connected over a network, like for instance a scalp mounted device that can transmit the measured data, also remotely, to an EEG/NIRS device; a camera connected to a remote processing unit, etc. In this way, it is possible to collect data for the learning process, wherein the emotion related data and the activity related data are close to each other in time, ideally or optionally refer to the same point in time within a certain tolerance or threshold. The assisting unit 190 provides driving assistance of the vehicle based on the estimated emotion.

Preferably, the driving assistance may include an active control of the vehicle by the assisting unit during driving: for instance, if the estimated emotion is found to be associated to an hazardous situation, the control unit (or any other unit suitable for automatically or semi automatically driving the vehicle) may act on components of the vehicle like the brakes to slow down the vehicle, and/or on the steering wheel to take over control (e.g. an automatic pilot) or to stop the vehicle. Preferably, the driving assistance may include providing the driver of the vehicle with at least a feedback during driving. For instance, when the emotion estimation is associated with an hazardous situation, the assisting unit may provide, as driving assistance, a message (as an example of the feedback) to the driver suggesting to make a stop and take a rest. Another example of feedback is represented by a sound, melody, music, or audio message in general; in this way, the driver may be alerted so that the hazardous situation is avoided. In general, therefore, the feedback may be represented for instance by one or more messages (in the form of text, audio, and/or video, etc.), or one or more stimuli signals induced on the subject. Other types of feedback are of course suitable.

Preferably, the apparatus 100 includes a cognitive state estimating unit for determining a cognitive state of the subject based on further information indicating an activity of the subject, wherein the assisting unit is further configured to provide driving assistance driving of the vehicle further based on the cognitive state. In this way, the overall mental state can be more accurately assessed based on both the estimated cognition and the estimated emotion; thus, a safer driving can be obtained (since the vehicle driving is assisted when it is really needed) thanks to a more accurate estimation of the mental state.

Preferably, the assisting unit is configured to provide a degree of driving assistance inversely corresponding to at least one amongst a degree of estimated arousal and a degree of estimated valence included in the estimated current emotion. By degree of driving assistance it is meant the extent of intervention provided on the driver and/or on the vehicle. For instance, a higher degree of driving assistance includes: providing driving assistance more frequently (e.g. with a higher frequency, or at shorter intervals) by more frequently actively intervening (on the vehicle components) and/or more frequently providing feedback; and/or providing more active intervention than compared to feedback; with shorter delays from when a condition for providing driving assistance is determined (e.g. short delay from when the estimated emotion is found to be associated to a hazardous situation). A degree of estimated arousal indicates how large is the value of the estimated arousal included in the estimated emotion; correspondingly, a degree of estimated valence indicates how large is the value of the estimated valence included in the estimated emotion. In one example, when the value for the estimated arousal and/or the value for the estimated valence decrease (i.e. their respective degree decreases), then the level of intervention is increased (i.e. the degree of intervention is increased), and vice versa. The relationship between the assistance degree and arousal/valence degree can be inversely proportional, or non-linear as found for instance by experiments.

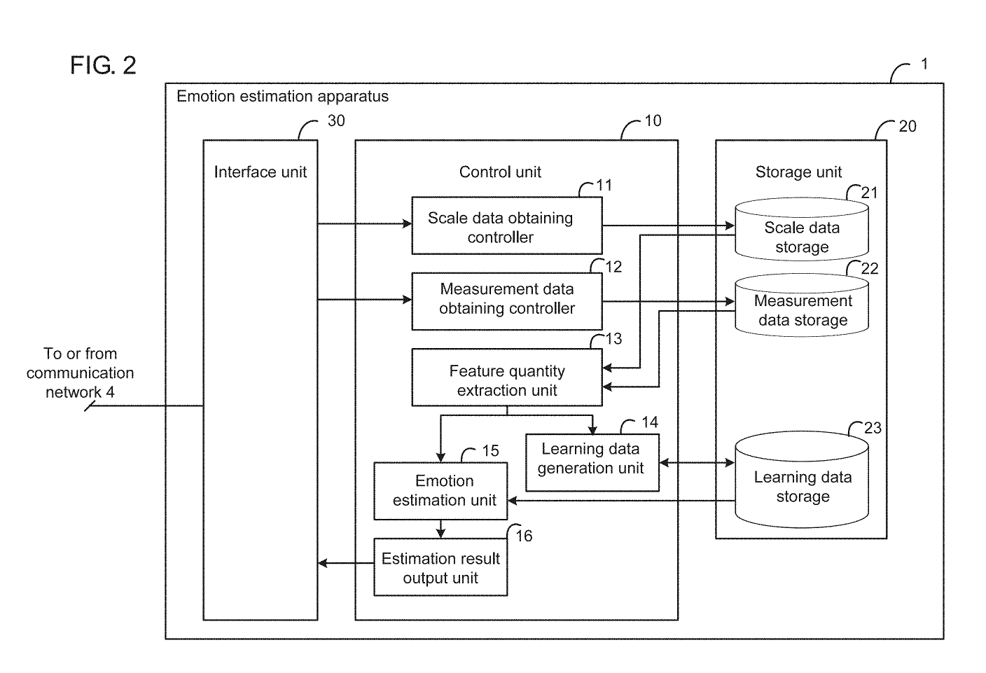

Optionally, the apparatus of the present embodiment can also be represented like in FIG. 14B: as to its illustration, we refer to the below description of FIG. 2, noting that schematic blocks or units having the same reference signs have the same function in both figures, and that the storage unit 120, the learning data generation unit 114, and the emotion estimation unit 115 of FIG. 14B correspond to the units 20, 14, and 15, respectively. Also, other considerations made below with reference to FIG. 2 also apply to FIG. 14B. We note however that the assisting unit 190 is not included in FIG. 2, since that embodiment is directed to how to obtain the emotion estimation, which is suitable for use in different applications like assisted driving, controlling of a manufacturing line, health care support, etc.

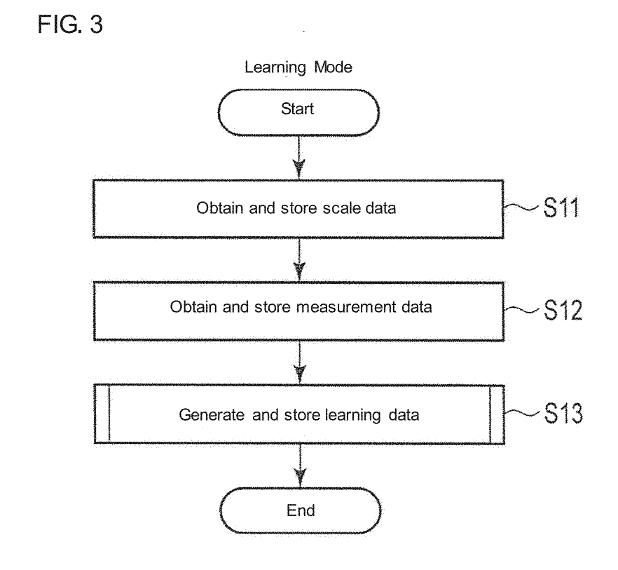

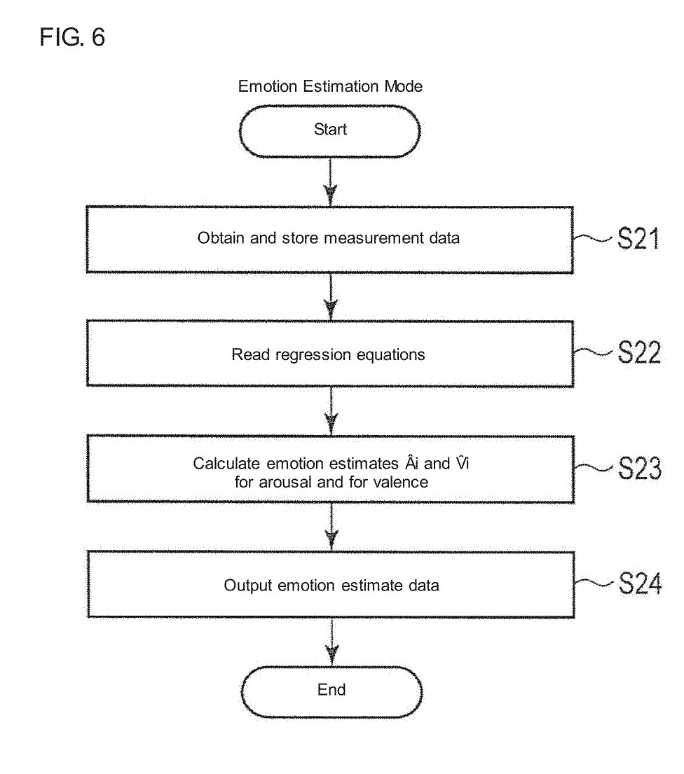

An operation of the apparatus for assisting driving according to the present embodiment is now described with reference to FIG. 14C. At step S1110, information indicating an emotion of a subject, and information indicating an activity of the subject are stored. At step S1113, learning is performed of data representing a relationship between the information indicating the emotion of the subject obtained by the first obtaining unit, and the information indicating the activity of the subject obtained by the second obtaining unit and store the learning data into a memory. At step S1123, after the learning data is generated, it is estimated a current emotion of the subject based on information indicating a current activity of the subject obtained by the second obtaining unit, and the learning data stored in the memory. Then, at step S1190, it is provided a driving assistance of the vehicle based on the estimated current emotion. It is noted that an example of step 1110 is provided for instance by the combination of steps S11 and S12 (of FIG. 3) below illustrated. Also, an example of step S1113 is provided by below step S13 of FIG. 3. Further, an example of step S1123 is provided by step S23 of FIG. 6 below illustrated. Other operations or method steps are immediately evident from the respective description of the apparatus according to the present embodiment and the below embodiment 2. Further, in this and other methods herein described, steps are defined like storing, generating, estimating, controlling, etc. It is however noted that such steps (or any combination of them) may also be caused or induced by a remote device, like for instance by a client computer or a portable terminal, on another device (like for instance a server, localized or distributed) that correspondingly performs the actual step. Thus, the mentioned steps are to be understood also as causing to store, causing to generate, causing to estimate, cause to control, etc., such that any of their combination can be caused or induced by a device remote to the device actually performing the respective step.

### Embodiment 2—Emotion Estimation Apparatus

### Structure

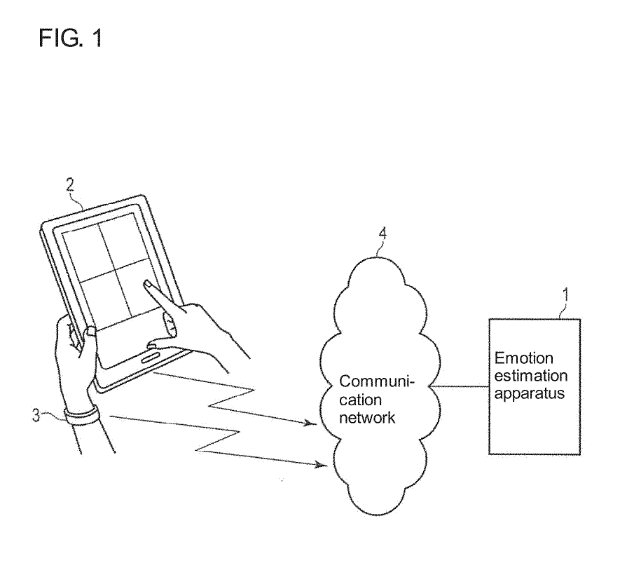

FIG. 1 is an overview of a system including an emotion estimation apparatus according to one embodiment of the present invention. The emotion estimation system according to this embodiment includes an emotion estimation apparatus 1, an emotion input device 2, and a measurement device 3. The emotion input device 2 and the measurement device 3 can communicate with the emotion estimation apparatus 1 through a communication network 4.

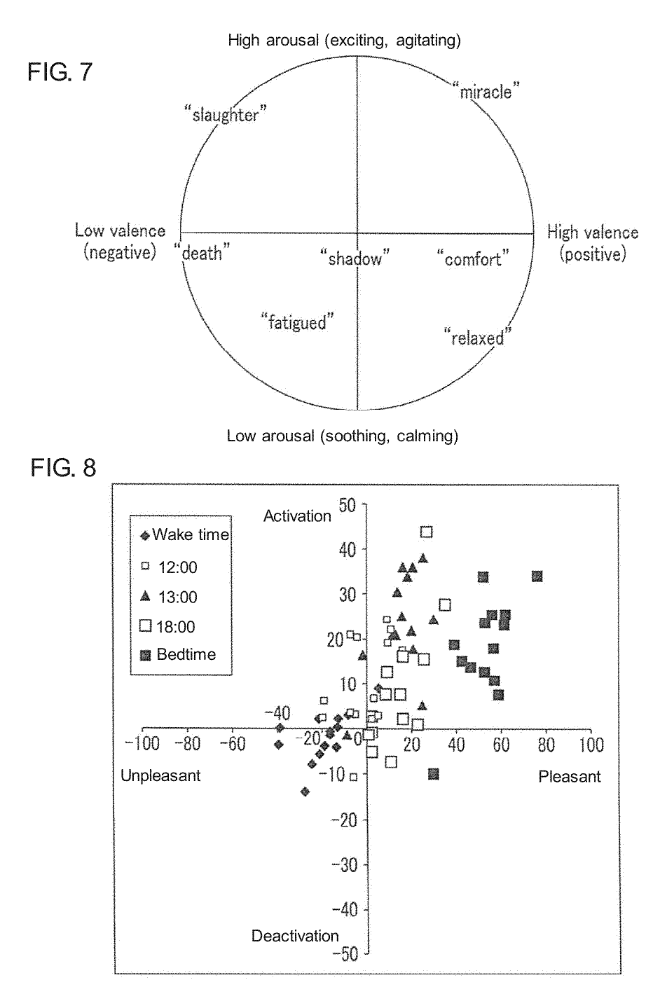

The emotion input device 2, which is for example a smartphone or a tablet terminal, displays an emotion input screen under control with application programs. The emotion input screen shows emotions using a two-dimensional coordinate system with emotional arousal on the vertical axis and emotional valence on the horizontal axis. When a subject plots the position corresponding to his or her current emotion on the emotion input screen, the emotion input device 2 recognizes the coordinates indicating the plot position as information indicating the emotion of the subject. This technique of expressing the emotions using arousal and valence on the two-dimensional coordinate system is known as the Russell's circumplex model. FIG. 7 schematically shows this model.

FIG. 8 is a diagram showing example input results of emotion at particular times obtained through the emotion input device 2. The arousal indicates the emotion either being activated or deactivated and the degree of activation to deactivation, whereas the valence indicates the emotion either being pleasant or unpleasant and the degree of being pleasant to unpleasant.

The emotion input device 2 transforms the position coordinates detected as the emotion information to the arousal and valence values and the information about the corresponding quadrant of the two-dimensional arousal-valence coordinate system. The resultant data, to which the time stamp data indicating the input date and time is added, is transmitted as emotion input data (hereinafter referred to as scale data) to the emotion estimation apparatus 1 through the communication network 4 using a wireless interface.

The measurement device 3 is, for example, incorporated in a wearable terminal, and is mounted on a wrist of the subject as shown in FIG. 1. The measurement device 3 measures information indicating human activity correlated with human emotions. The information indicating human activity includes vital signs and motion information. To measure the vital signs and the motion information, the measurement device 3 includes various vital sign sensors and motion sensors. Examples of the vital sign sensors and the motion sensors include any combination (any combination in the present text includes one single element of a list of elements or two or more elements of the list) of sensors for measuring heart electrical activity H, skin potential activity G, motion BM, and an activity amount Ex. Also, any combination of blood pressure, heart rate, pulse, respiration rate, depth of respiration, body temperature, and eye-blink rate, which are measured by the known sensors, may be used as the activity information. When the emotion estimation is applied to the subject such as a driver of a vehicle (e.g. embodiment 1), a worker in a manufacturing line (see e.g. embodiment 3), or a user of a healthcare management system (see e.g. embodiment 4), it is preferable to use the appropriate sensors which may not prevent the necessary movements of the subject and can be used during the operations or usual activities. Using the above activity information to be obtained or measured with the appropriate sensors makes it possible to estimate the real-time emotions and utilize the estimation results with minimum delays, and without impairing the usual activities of the subject (e.g. when the subject is interacting with a machine).

The heart electrical activity sensor measures the heart electrical activity H of the subject in predetermined cycles or at selected timing to obtain the waveform data, and outputs the measurement data. The skin potential activity sensor, which is for example a polygraph, measures the skin potential activity G of the subject in predetermined cycles or at selected timing, and outputs the measurement data. The motion sensor, which is for example a triaxial acceleration sensor, measures the motion BM, and outputs the triaxial acceleration measurement data. The sensor for measuring the activity amount Ex, which is an activity sensor, outputs the measurement data indicating the intensity of physical activity (metabolic equivalents, or METs) and the amount of physical activity (exercise). Reference is also made to the above discussion about obtaining activity information by means of activity sensor(s) set according to respective activity sensor configuration(s).

Another sensor for measuring vital signs correlated with human emotions is an eye movement (EM) sensor. This sensor is a small image sensor, and is mounted on, for example, a frame of glasses or goggles.

Reference is made above to the example of an emotion input device 2 wherein the emotion is inserted directly by a user; this is however not indispensable, and it can be in fact omitted, in which case the emotion can be (indirectly, it can be said) obtained by means of the measurement device 3, which can include in fact suitable sensors and/or measurement devices as explained above or with reference to FIGS. 12 and 13. Preferably, the emotional state is measured by devices capable of determining such emotional state with high accuracy, regardless of how large and/or complex such devices are, further preferably by devices a higher accuracy in determining the emotional state than compared with wearable devices used for the same determination. In other words, when using sufficiently complex devices (like e.g. a PET/f MRI device) the emotional state can be determined with sufficient accuracy also without the input from the user. In certain cases, also using a scalp mounted EEG/NIRS device, it is also possible to determine the emotional state with sufficient accuracy without the user input. As previously illustrated, however, there are different sensors and/or configuration of sensors for obtaining measurement of physiological parameters related to the emotional state of the subject. Also, the emotion can be obtained by a combination of the above, e.g. by combining information entered directly by the person and information acquired via sensor(s).

In this and other embodiments, the information indicating an emotion of a subject is preferably expressed in a two-dimensional coordinate system having a first axis representing an arousal state and a second axis representing a valence state, and the estimated current emotion is output as values corresponding to the arousal state and the valence state. Further, as also herein described, output values may be an arousal value and a valence value (the coordinates in the two-dimensional system), or the variation in the arousal value and valence value. In other words, the estimated emotions are estimated as sets of arousal value and valence value, and thus the emotional state can be expressed by coordinates in the two dimensional coordinate system. This configuration makes it possible to estimate wide varieties of emotional states in an objective and repeatable way (technically representable in a computer system), including the emotional states unable to be defined in verbal expressions such as “excited” “depressed” “happy” “sad” (which would be in fact not be easily manageable in a computer system), and to track the continuous changes in the emotional states. Therefore, the estimation accuracy improves and more detailed and delicate control can be executed in the system using the estimated emotion, as it become evident when applying this to the herein described embodiments.

The measurement device 3 adds the time stamp data indicating the measurement date and time to the measurement data obtained with each sensor. The measurement device 3 transmits the measurement data to the emotion estimation apparatus 1 through the communication network 4 using a wireless interface. The measurement device 3 may not be incorporated in a wearable terminal, and may be mountable on clothes, a belt, or a helmet.

The wireless interfaces used by the emotion input device 2 and the measurement device 3 to transmit the measurement data comply with, for example, low-power wireless data communication standards such as wireless local area networks (WLANs) and Bluetooth (registered trademark). The interface between the emotion input device 2 and the communication network 4 may be a public mobile communication network, or a signal cable such as a universal serial bus (USB) cable.

The emotion estimation apparatus 1 is, for example, a personal computer or a server computer with the structure described below. FIG. 2 is a block diagram showing the functional components of the apparatus. The emotion estimation apparatus 1 includes a control unit 10, a storage unit 20 (also corresponding to the storage unit 120 of FIG. 14B), and an interface unit 30.

The interface unit 30, which allows data communication in accordance with a communication protocol defined by the communication network 4, receives the scale data and the measurement data transmitted from the emotion input device 2 and the measurement device 3 through the communication network 4. The interface unit 30 also includes an input-output interface function for receiving data input from an input device, such as a keyboard or a mouse, and outputting display data input from the control unit 10 to a display (not shown) on which the data will appear.

The storage unit 20 is a storage medium, and is a readable and writable non-volatile memory, such as a hard disk drive (HDD) or a solid state drive (SSD). The storage unit 20 includes a scale data storage 21, a measurement data storage 22, and a learning data storage 23 as storage areas used in the embodiments.

The scale data storage 21 stores scale data representing the emotion of the subject transmitted from the emotion input device 2. The measurement data storage 22 stores measurement data transmitted from the measurement device 3. The learning data storage 23 stores learning data generated by the control unit 10.

The control unit 10 includes a central processing unit (CPU) and a working memory. The control unit 10 includes a scale data obtaining controller 11, a measurement data obtaining controller 12, a feature quantity extraction unit 13, a learning data generation unit 14 (also corresponding to the unit 114 of FIG. 14B), an emotion estimation unit 15 (also corresponding to the unit 115 of FIG. 14B), and an estimation result output unit 16 as control functions used in the embodiments. Each of these control functions is implemented by the CPU executing the application programs stored in program memory (not shown).

The scale data obtaining controller 11 implements the function of a first obtaining unit in cooperation with the interface unit 30. When an operation for inputting emotions is performed with the emotion input device 2, the scale data obtaining controller 11 obtains the scale data transmitted from the emotion input device 2 through the interface unit 30, and stores the obtained scale data in the scale data storage 21.

The measurement data obtaining controller 12 implements the function of a second obtaining unit in cooperation with the interface unit 30. The measurement data obtaining controller 12 obtains the measurement data transmitted from the measurement device 3 through the interface unit 30, and stores the obtained measurement data in the measurement data storage 22.

The feature quantity extraction unit 13 reads, from the scale data storage 21 and the measurement data storage 22, the scale data and the measurement data within each of the windows that are arranged at time points chronologically shifted from one another. The feature quantity extraction unit 13 extracts the feature quantities from the read scale data and the read measurement data, calculates the variation between the feature quantities, and transmits the calculation results to the learning data generation unit 14.

The windows each have a predetermined unit duration. The windows are defined in a manner shifted from one another by the above unit duration to avoid overlapping between chronologically consecutive windows, or in a manner shifted by a time duration shorter than the above unit duration to allow overlapping between chronologically consecutive windows. The unit duration of each window may be varied by every predetermined value within a predetermined range.

In a learning mode, the learning data generation unit 14 performs multiple regression analysis with correct values (supervisory data) being the variations among the feature quantities in the scale data for arousal and for valence within each window that are extracted by the feature quantity extraction unit 13 and variables being the variations among the feature quantities of the measurement data. This generates regression equations for arousal and for valence representing the relationship between the emotion of the subject and the feature quantities of measurement data. The learning data generation unit 14 associates the generated regression equations with window identifiers that indicate the time points of the corresponding windows, and stores the equations into the learning data storage 23 as learning data to be used for emotion estimation.

The learning data generation unit 14 generates, for each window, the regression equations for arousal and for valence for every change of the predetermined value in the unit duration of each window. The learning data generation unit 14 selects the window unit duration and the shift that minimize the difference between the sum of the time-series emotion estimates calculated using the generated regression equations and the sum of the correct values (supervisory data) of the emotion information included in the scale data, and transmits the selected window unit duration and the selected shift, and the corresponding regression equations to the emotion estimation unit 15.

In an emotion estimation mode after the learning data is stored, the emotion estimation unit 15 reads, for each window, the variations among the feature quantities extracted from the measurement data within each window from the feature quantity extraction unit 13, and also the regression equations for arousal and for valence corresponding to the window from the learning data storage 23. The emotion estimation unit 15 calculates the estimates of the emotional changes in arousal and in valence using the regression equations and the variations among the feature quantities in the measurement data, and outputs the calculation results to the estimation result output unit 16.

Based on the estimates of the emotional changes in arousal and in valence output from the emotion estimation unit 15, the estimation result output unit 16 generates information indicating the current emotional change in the subject and transmits the information, through the interface unit 30, to a relevant management apparatus.

### Operation

The operation of the emotion estimation apparatus 1 with the above structure will now be described in association with the operation of the overall system.

### (1) Learning Data Generation

Before the process for estimating the emotions of the subject, the emotion estimation apparatus 1 enters the learning mode and generates the learning data specific to the subject in the manner described below. FIG. 3 is a flowchart showing the procedure and its details.

For example, an operator of manufacturing equipment, who is a subject, inputs his or her current emotions with the emotion input device 2 at predetermined time intervals or at selected timing while working.

1-1: Input of Emotion Information by Subject, Measurement of Vital Signs and Motion Information about Subject Activity, and Collection of Input Data and Measurement Data

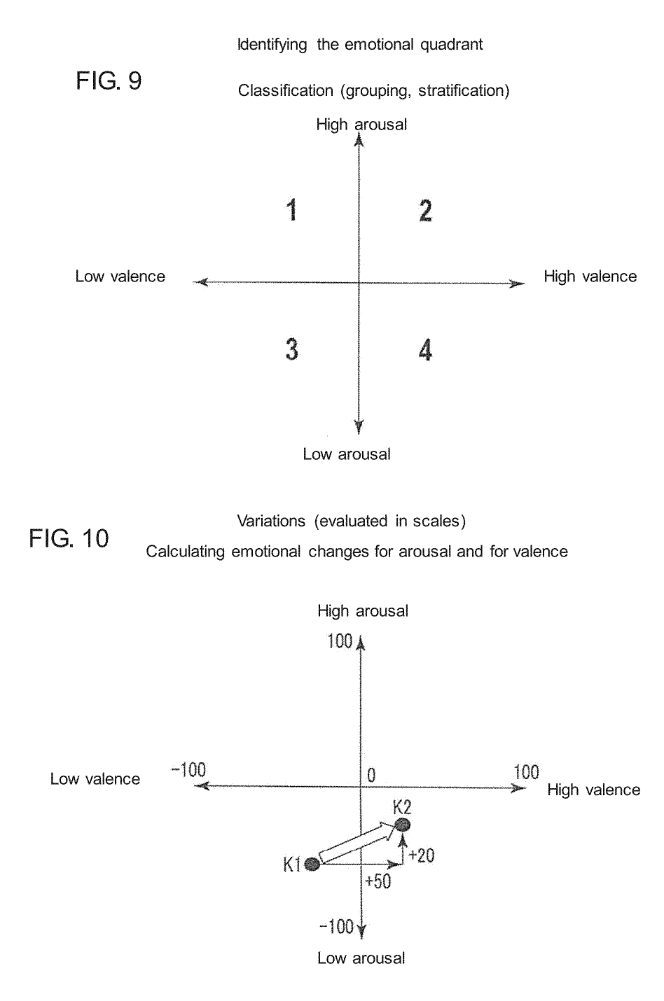

As described above, the emotion input device 2 displays the emotion of the subject in the two-dimensional coordinate system for emotional arousal and emotional valence, and detects the coordinates of a position plotted by the subject on the two-dimensional coordinate system. The two-dimensional coordinate system used in the emotion input device 2 has the four quadrants indicated by 1, 2, 3, and 4 as shown in FIG. 9, and the arousal and valence axes each representing values from −100 to +100 with the intersection point as 0 as shown in FIG. 10. The emotion input device 2 transforms the detected coordinates to the information about the corresponding quadrant and to the corresponding values on both the arousal and valence axes. The emotion input device 2 adds the time stamp data indicating the input date and time to the resultant information, and transmits the data to the emotion estimation apparatus 1 as scale data.

In parallel with this, the measurement device 3 measures the heart electrical activity H, the skin potential activity G, the motion BM, and the activity amount Ex of the working subject at predetermined time intervals, and transmits the measurement data to the emotion estimation apparatus 1 together with the time stamp data indicating the measurement time. Additionally, the eye movement EM of the subject is measured by an image sensor (not shown), and the measurement data is also transmitted to the emotion estimation apparatus 1 together with the time stamp data.

In step S11, the emotion estimation apparatus 1 receives the scale data transmitted from the emotion input device 2 through the interface unit 30 as controlled by the scale data obtaining controller 11, and stores the received scale data into the scale data storage 21. In step S12, the emotion estimation apparatus 1 receives the measurement data transmitted from the measurement device 3 and the image sensor through the interface unit 30 as controlled by the measurement data obtaining controller 12, and stores the received measured data into the measurement data storage 22.

### 1-2: Learning Data Generation

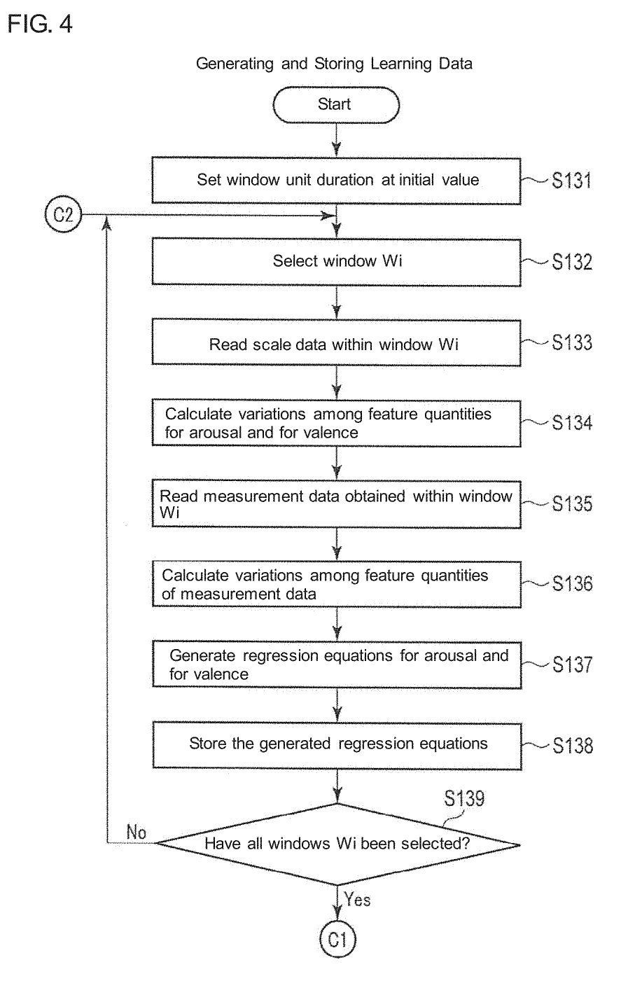

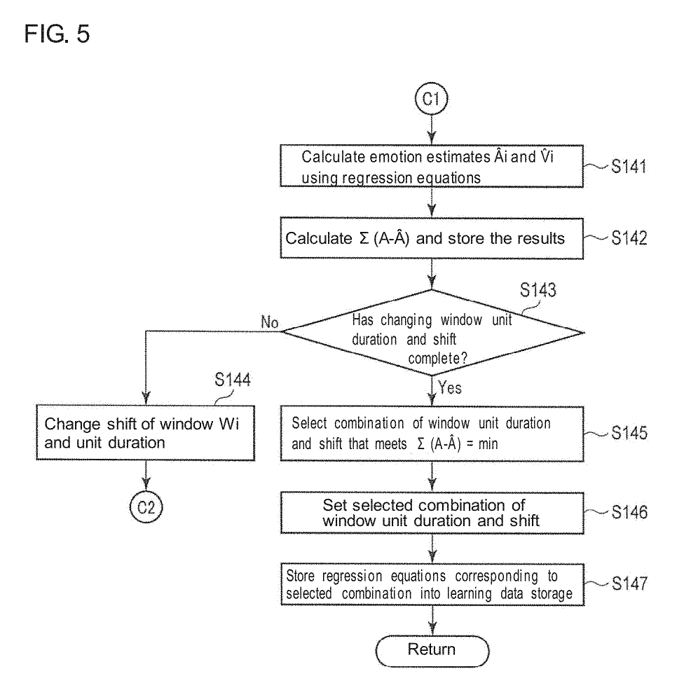

In step S13, when the scale data and the measurement data accumulate for a predetermined period (e.g., one day or one week), the emotion estimation apparatus 1 generates learning data as controlled by the feature quantity extraction unit 13 and the learning data generation unit 14 in the manner described below. FIGS. 4 and 5 are flowcharts showing the procedure and its details.

In step S131, the unit duration of the window Wi (i=1, 2, 3, . . . ) is set at an initial value. In step S132, the first window (i=1) is selected. In step S133, the feature quantity extraction unit 13 reads a plurality of sets of scale data within the first window from the scale data storage 21. In step S134, the feature quantity extraction unit 13 calculates the variations among the feature quantities for arousal and for valence. For example, when scale data K1 and scale data K2 are input within the unit duration of one window as shown in FIG. 10, the variations are calculated as the change from the third to the fourth quadrant, and as the increment of 20 (+20) for arousal and the increment of 50 (+50) for valence. Even for a change to a diagonally opposite quadrant, for example, for a change from the third to the second quadrant, the variations among the resultant feature quantities may be calculated for arousal and for valence.

In step S135, the feature quantity extraction unit 13 reads all the items of measurement data obtained within the unit duration of the first window, which are the heart electrical activity H, the skin potential activity G, the motion BM, the activity amount Ex, and the eye movement EM, from the measurement data storage 22. In step S136, the feature quantity extraction unit 13 extracts the feature quantities from the measurement data.

For example, the heart electrical activity H has the feature quantities that are the heartbeat interval (R-R interval, or RRI), and the high frequency components (HF) and the low frequency components (LF) of the power spectrum of the RRI. The skin potential activity G has the feature quantity that is the galvanic skin response (GSR). The eye movement EM has the feature quantities that are the eye movement speed and the pupil size. The motion BM has feature quantities including the hand movement speed. The hand movement speed is calculated based on, for example, the triaxial acceleration measured by the triaxial acceleration sensor. The activity amount Ex has the feature quantities that are the intensity of physical activity (METs) and the exercise (EX). The exercise (EX) is calculated by multiplying the intensity of physical activity (METs) by the activity duration.

The feature quantity extraction unit 13 calculates the variations among the extracted feature quantities that are the heart electrical activity H, the skin potential activity G, the biological motion BM, the activity amount Ex, and the eye movement EM within the unit duration of the window.

In step S137, the learning data generation unit 14 generates learning data for arousal and learning data for valence based on the variations calculated in step S134 between the scale data feature quantities and the variations calculated in step S136 between the measurement data feature quantities. For example, the learning data generation unit 14 performs multiple regression analysis using the variations among the scale data feature quantities for arousal and for valence as supervisory data, and the variations among the measurement data feature quantities as independent variables, which are primary indicators. The learning data generation unit 14 then generates regression equations for arousal and for valence representing the relationship between the change in the emotion of the subject and the change in the vital signs and motion information.

The regression equations corresponding to the i-th window are as follows:

Âi=f(α1Hi, α2Gi, α3EMi, α4BMi, α5Exi), and

\hat{V}i=f(α1Hi, α2Gi, α3EMi, α4BMi, α5Exi)

where Âi is the estimate of the arousal change, \hat{V}i is the estimate of the valence change, α1, α2, α3, α4, and α5 are the weighting coefficients for the feature quantities of the measurement data items Hi, Gi, EMi, BMi, and Ex, and f is the sum of the indicators obtained from the feature quantities of the measurement data Hi, Gi, EMi, BMi, and Ex, which are primary indicators. The weighting coefficients may be determined by using, for example, the weighted average based on the proportions in the population data obtained in the learning stage.

In step S138, the learning data generation unit 14 stores the generated regression equations for arousal and for valence corresponding to the i-th window into the learning data storage 23. In step S139, the learning data generation unit 14 determines whether all the windows Wi have been selected for generating regression equations. When any window remains unselected, the processing returns to step S132, where the unselected window is selected, and the learning data generation processing in steps S133 to S139 is repeated for the next selected window.

### 1-3: Determination of Optimum Window Unit Duration and Shift

In general, the window unit duration may be determined or changed in several ways (with this regard, it is noted that even if the window is named predetermined, it means that it can be conveniently determined; thus, the expression predetermined window unit is also interchangeable with determined window unit).

For example, the predetermined unit duration of a window may be determined (or calculated) on the basis of at least one amongst:

(i) obtained activity information (obtained by the second obtaining unit) indicating an activity state of the subject, and
(ii) an interaction time interval indicating a time length for an interacting operation between a device coupled to the apparatus and the subject.

In case (i), in other words, depending on the obtained information about the activity of the subject (e.g. the type of activity, intensity, etc.), the unit duration can be changed. As further illustration, the unit duration of the window may be changed based on the activity information item to be used in the emotion estimation. For example, a table which defines the unit duration suitable for each type of the activity information items, determined by experiment or the like, is stored in a memory, and the unit duration corresponding to the activity information to be used in the emotion estimation is read out form the table. In a case where the multiple activity information items are used in the emotion estimation, the unit duration may be determined based on the activity information item having the highest priority or weight in the regression equation for estimating the emotion. The unit duration may be adjusted based on the characteristic of each user, that is, the individual baseline level of the activity information item. In this case, the baseline level of the user may be determined by comparing the obtained activity information item with threshold(s), and the unit duration may be determined based on the determined baseline level such that the smaller unit duration is set for the larger (faster) heart rate base line, for example. The baseline level may be determined in consideration of the physical condition of the user.

Case (ii) above may be considered, for instance, when the subject interacts with a device, the device being for example a vehicle (see e.g. embodiment 1), a component of a manufacturing line (see e.g. embodiment 3), or a healthcare support providing device (e.g. a device providing in the form of a feedback a stimulus to the subject, see also embodiment 4 below). In these scenarios, the estimated emotion may be used to control the interaction of the subject with the device by means of the apparatus of this and other embodiments. In particular, in these scenario, the window unit duration may be determined to comply with a time interval representing a typical interaction interval between the subject and the device. For instance, in the scenario of a manufacturing line, the interaction time interval may be the cycle time for producing one item by means of the line, or a cycle time for a manufacturing line component to perform the operation for which it intended (e.g. time needed to machine one piece, etc.). In the case of a vehicle, the interaction interval may be preset, or variable depending on the hour of the day, of variable depending on the type of road driven by the vehicle (e.g. different intervals depending on country road or highway, on straight road or road with many turns, etc.). In the case of a healthcare support device, the interacting time interval may be linked to physiological parameter of the subject (body temperature, level of activity, etc.)

The feature quantity extraction unit 13 and the learning data generation unit 14 change the window unit duration by every predetermined value and the chronological shift of the window by every predetermined amount to determine the optimum window unit duration and the optimum shift. Of all the combinations of the unit durations and the shifts, the learning data generation unit 14 selects a combination that minimizes the difference between the emotion estimates obtained using the regression equations and the emotion information correct values input through the emotion input device 2. The learning data generation unit 14 then sets, for the emotion estimation mode, the selected window unit duration and the selected shift, as well as the regression equations generated for this combination. In scenario (ii), therefore, it is possible to arrive at a much more accurate estimation, which is directly linked to the type of interaction between man and machine. As a consequence of the more accurate estimation, as already explained, it is also possible to provide a better driving assistance, or (as later described) an improved productivity on the manufacturing line (see e.g. embodiment 3), or improved health conditions (see e.g. embodiment 4).

The above processing will now be described. FIG. 5 is a flowchart showing the procedure and its details.

In step S141, the learning data generation unit 14 calculates the emotion estimates Âi and \hat{V}i using the regression equations generated for each window Wi, and computes the sum of the calculated estimates Âi as Â and the sum of the calculated estimates \hat{V}i as \hat{V}. In step S142, the learning data generation unit 14 calculates the differences between the sums of the emotion estimates Â and \hat{V}, and the sums of the true values A and V of the emotion information input through the emotion input device 2 in the manner described below.

Σ(A−Â) and Σ(V−\hat{V})

The calculation results are stored into the learning data storage 23. For simplifying the flowchart, FIG. 5 only shows Σ(A−Â).

In step S143, the learning data generation unit 14 determines whether changing the window unit duration and the shift has been complete, or in other words, whether regression equations have been generated for all combinations of the window unit durations and the shifts. When this process is incomplete, the processing advances to step S144, in which the unit duration and the shift of the window Wi is changed by the predetermined amount. The processing then returns to step S132 shown in FIG. 4, and then the processing in steps S132 to S143 is performed. In this manner, the processing in steps S132 to S144 is repeated until the regression equations are generated for all combinations of the window unit durations and the shifts.

When the regression equations have been generated for all the combinations of the window unit durations and the shifts, the learning data generation unit 14 compares the differences, calculated for all the combinations of the window unit durations and the shifts, between the sums of the emotion information true values A and V, and the sums of the emotion estimates Â and \hat{V}, which are Σ(A−Â) and Σ(V−\hat{V}), in step S145. The learning data generation unit 14 then selects the combination of the window unit duration and the shift that minimizes the values of Σ(A−Â) and Σ(V−\hat{V}).

In step S146, the learning data generation unit 14 sets the selected combination of the window unit duration and the shift in the feature quantity extraction unit 13. In step S147, the learning data generation unit 14 stores the regression equations corresponding to the selected combination into the learning data storage 23. The learning data generation process ends.

### (2) Emotion Estimation

When the learning data generation is complete, the emotion estimation apparatus 1 uses the learning data to estimate the emotions of the working subject. FIG. 6 is a flowchart showing the procedure and its details.

The measurement device 3 measures any of or any combination of the heart electrical activity H, the skin potential activity G, the eye movement EM, the motion BM, and the activity amount Ex of the working subject at predetermined time intervals or predetermined timing, and transmits the measurement data to the emotion estimation apparatus 1. Preferably, in the context of emotional state estimation, the devices used for such measurements need not be highly accurate as those used for obtaining measurement data used for the generation of the learning data. Further preferably, the measurement device 3 in the context of emotion estimation are wearable devices like any sensor wearable by a person and capable preferably of calculating and delivering the result of the measure to another device (like a smartphone).

In step S21, the emotion estimation apparatus 1 receives the measurement data transmitted from the measurement device 3 and the image sensor through the interface unit 30 as controlled by the measurement data obtaining controller 12, and stores the received data into the measurement data storage 22. The feature quantity extraction unit 13 included in the emotion estimation apparatus 1 reads the measurement data from the measurement data storage 22 with the window unit duration determined in the learning data generation process described above, and extracts the feature quantities from the measurement data. The extracted feature quantities are the same as those extracted in the learning mode, and will not be described in detail.

In step S22, the emotion estimation apparatus 1 reads, from the learning data storage 23 as controlled by the emotion estimation unit 15, the regression equations for arousal and for valence corresponding to the time period in which the measurement data is obtained. In step S23, the emotion estimation apparatus 1 calculates the emotion estimates Âi and \hat{V}i for the subject in the time period in which the measurement data is obtained using the regression equations and the feature quantities of the measurement data. In step S24, the estimation result output unit 16 generates display data representing the current emotions of the subject based on the calculated emotion estimates Âi and \hat{V}i for arousal and for valence, and transmits the display data to, for example, a manager's terminal, on which the data will appear.

The manager (or the apparatus, via the control unit) then instructs the subject to rest or continue working based on the estimation results associated with the emotion of the subject appearing on the terminal.

### Advantageous Effects

As described in detail above, in one embodiment, regression equations for estimating emotional changes in arousal and in valence are generated in the learning mode by multiple regression analysis with supervisory data being information indicating the emotion of the subject input through the emotion input device 2, and variables being the feature quantities obtained from the measurement data items by the measurement device 3 in the same time period, which are the heart electrical activity H, the skin potential activity G, the eye movement EM, the motion BM, and the activity amount Ex of the subject. The emotional changes of the subject are estimated using the regression equations and the changes in the feature quantities of the measurement data items, which are the heart electrical activity H, the skin potential activity G, the eye movement EM, the motion BM, and the activity amount Ex of the subject measured by the measurement device 3.

In the estimation mode, the current emotional changes of the subject can thus be estimated in real time based on the measurement data, which includes the subject's vital signs and motion information, and the regression equations preliminarily generated as the learning data. The emotional changes of the subject can be estimated without monitoring external events, such as the environment conditions around the subject. This relatively simple structure without any component for monitoring external events around the subject has a wide range of applications.

Additionally, emotional changes are expressed simply and accurately using the quadrants of the two-dimensional arousal-valence coordinate system and the variations for arousal and for valence.

Further, the emotional changes of the subject are estimated precisely in each time period using regression equations generated for each of the windows that are arranged at time points chronologically shifted time from one another to estimate the emotional changes based on the time-series measurement data. In addition, the windows are defined using the window unit duration and the shift that are changed by every predetermined value. Regression equations are generated for all combinations of the unit durations and the shifts. The combination of the window unit duration and the shift that minimizes the difference between the emotion estimates obtained from these regression equations and the emotion true values input through the emotion input device 2 is selected and set. The emotional changes of the subject can thus be estimated accurately.

### Embodiment 3—Apparatus for Controlling a Manufacturing Line

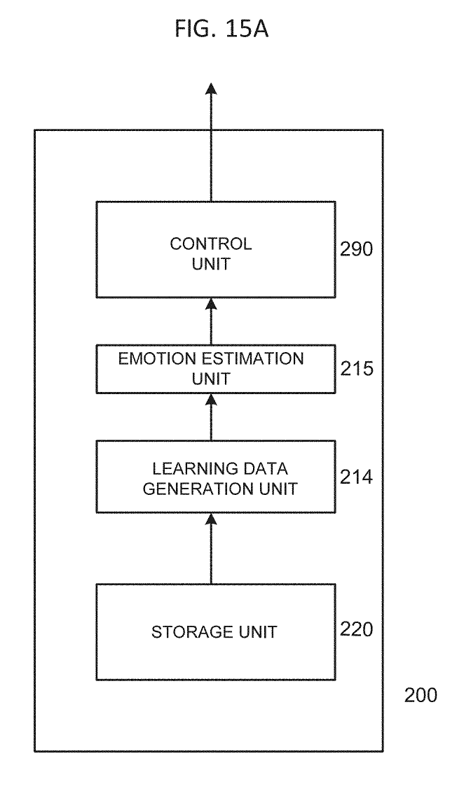

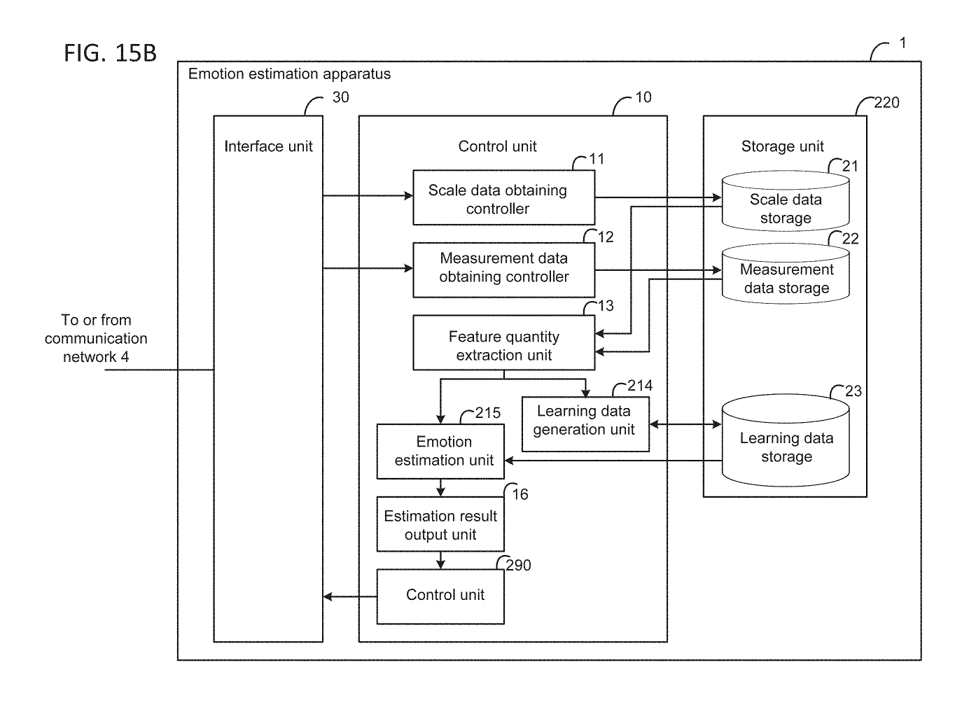

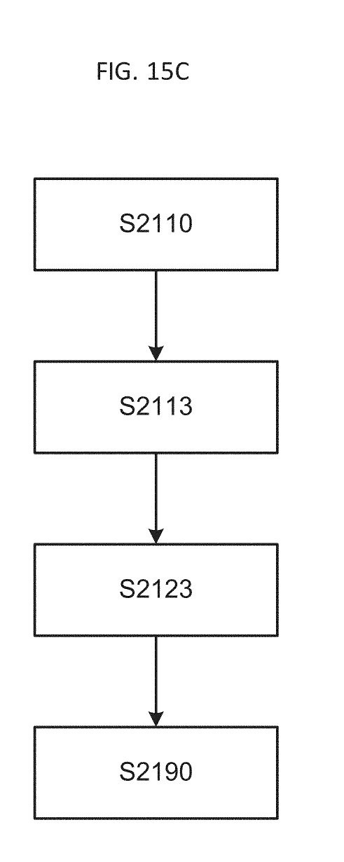

In embodiment 1, an apparatus for assisting in driving a vehicle has been presented, which may preferably include some or all of the features of embodiment 2 describing an emotion estimation apparatus used to estimate the emotion used for determining the driving assistance. Embodiment 3 is directed to an apparatus for controlling a manufacturing line, wherein the manufacturing line is controlled on the basis of an estimated emotion. Thus, similarly to embodiment 1, also in embodiment 3 the estimated emotion can be obtained by means of the device of embodiment 2, such that part or all of the features of embodiment 2 (and their operation, methods, etc.) can be optionally included into embodiment 3. Embodiment 3 will now be described with reference to FIG. 15A, showing an apparatus 200 for controlling a manufacturing line, wherein the apparatus comprises a storage unit 220, a learning data generation unit (214), an emotion estimation unit 215, and a control unit 290.

The storage unit 220 stores information indicating an emotion of a subject, and information indicating an activity of the subject. The subject is for example, a worker or an operator working on or interacting with the manufacturing line during its operation. The learning data generation unit 214 generates learning data representing a relationship between the stored information indicating the emotion of the subject (preferably obtained by a first obtaining unit, see embodiment 2), and the stored information indicating the activity of the subject (preferably obtained by a second obtaining unit, see embodiment 2) and store the learning data into a memory. The emotion estimation unit 215 estimates, after the learning data is generated, a current emotion of the subject based on information indicating a current activity of the subject obtained by an obtaining unit (the second obtaining unit; this is acquired or obtained in correspondence of the estimation), and the learning data stored in the memory. The control unit 290 controls the manufacturing line based on the estimated current emotion.

The manufacturing line may comprise one or more components including for example a machine (including tooling machines, molding tools, industrial ovens, for apparatuses for manufacturing semiconductors, etc.), a parts feeder, a robot, a controller for controlling a machine, etc. In general, a component may be automatic (e.g. once programmed, it operates without direct intervention of the worker, but may optionally still require interaction with a worker who interacts as supervisor), semi-automatic (i.e. partially operated by the worker), or manual; in general, the component interacts with the worker/operator when the manufacturing line is operating.

Further, the control unit of the present embodiment may perform any combination amongst controlling the speed of movement of a manufacturing line component and controlling the speed of operation of a manufacturing line component. For instance, when the estimated emotion is determined to have a predetermined value below a first threshold or within a first predetermined range, the control unit may for instance determine an intervention on the manufacturing line or on the component. For instance, one or more components may be stopped (e.g. temporarily), or its/their speed of operation and/or movement may be decreased. Alternatively or in addition, the worker may be provided with a feedback (similar to the case of the assisted driving apparatus). In this way, it can be avoided that the low emotional value, indicating an unsuitable mental state for working, may negatively affect productivity, or safety of the line or of the operator himself/herself. When the estimated emotion increases above a second threshold (not necessarily the same as the first threshold), or falls within a second predetermined range (different or partially overlapping with the first range), then one or more components may be controlled to re-start operation, or to increase speed of operation and/or movement.

Similarly to the other embodiments, also here the apparatus may optionally include a cognitive state estimating unit configured to determine a cognitive state of the subject based on further information indicating an activity of the subject, in which case the control unit is further configured to control the manufacturing line further based on the cognitive state. In this way, the mental state can be more accurately estimated on both the estimated cognitive state and estimated emotional state, such that the control unit can more effectively and accurately control the manufacturing line.

Further optionally, the control unit of the apparatus according to the present embodiment is configured to provide a degree of control of the manufacturing line inversely corresponding to at least one amongst a degree of estimated arousal and a degree of estimated valence included in the estimated current emotion. By degree is herein meant a level of production or productivity for the manufacturing line, e.g. dependent on the speed of operation/movement of one or more of its components, wherein the degree of production/productivity is changed in a way that is inverse to degree of the emotional state and/or degree of cognitive state (see also embodiment 1 with this regard).

Optionally, the apparatus of the present embodiment can also be represented like in FIG. 15B, noting that same reference signs indicate same components as in FIG. 2. Also, the storage unit 220, the learning data generation unit 214, and the emotion estimation unit 215 of FIG. 15B correspond to the units 20, 14, and 15, respectively. Thus, reference is made to embodiment 2 for further optional details, applicable also to the present embodiment.

An operation (method) of the apparatus for controlling a manufacturing line according to the present embodiment is now described with reference to FIG. 15C. At step S2110, information indicating an emotion of a subject, and information indicating an activity of the subject are stored. At step S2113, learning data are generated representing a relationship between the information indicating the emotion of the subject obtained by the first obtaining unit, and the information indicating the activity of the subject obtained by the second obtaining unit and store the learning data into a memory. At step S2123, it is estimated, after the learning data is generated, a current emotion of the subject based on information indicating a current activity of the subject obtained by the second obtaining unit, and the learning data stored in the memory. At step S2190, the manufacturing line is controlled based on the estimated current emotion.

It is noted that an example of step S2110 is provided for instance by the combination of steps S11 and S12 (of FIG. 3) above illustrated. Also, an example of step S2113 is provided by above step S13 of FIG. 3. Further, an example of step S2123 is provided by step S23 of FIG. 6 above illustrated. Other operations or method steps are immediately evident from the respective description of the apparatus according to the present embodiment and the above embodiments 1 and/or 2.

### Embodiment 4—Apparatus for Healthcare Support of a Subject

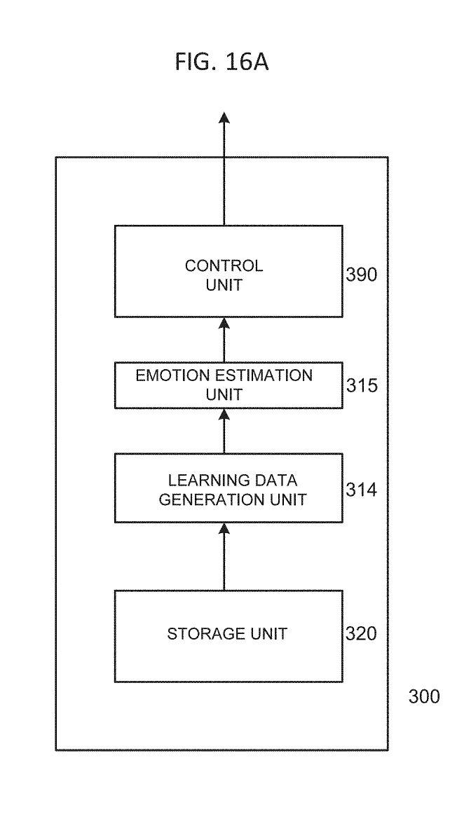

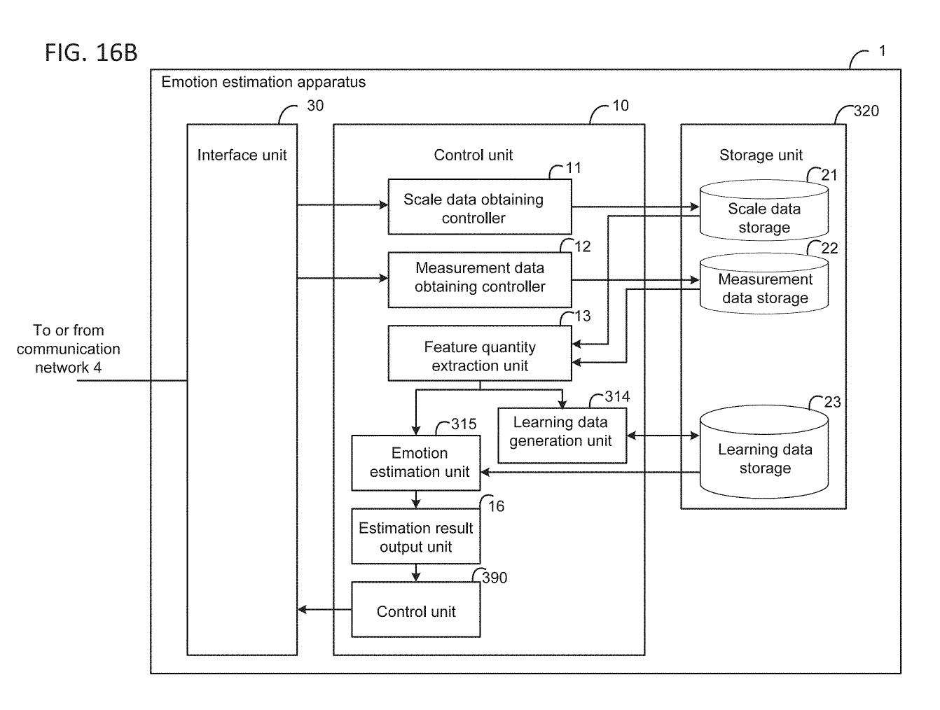

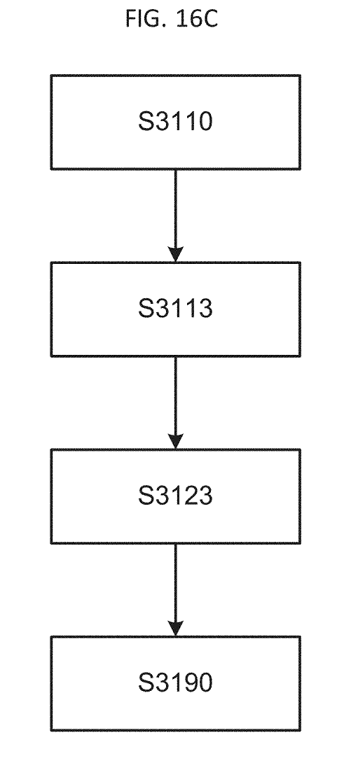

Present embodiment 4 is directed to an apparatus for healthcare support of a subject, wherein the apparatus provides the subject with a healthcare support feedback based on the estimated current emotion. By healthcare support for the subject is it herein meant that the device supports maintaining a certain health state/condition or improve the health state/condition of the subject. The subject can be for example any person of any age or sex. Similarly to embodiments 1 and 3, also in embodiment 4 the estimated emotion can be preferably obtained by means of the device of embodiment 2, such that part or all of the features of embodiment 2 (and their operation, methods, etc.) can be optionally included into embodiment 4. Embodiment 3 will now be described with reference to FIG. 16A, directed to an apparatus 300 for healthcare support of a subject, the apparatus comprising a storage unit 320, a learning data generation unit 314, an emotion estimation unit 315, and a control unit 390. The storage unit 320 stores information indicating an emotion of a subject, and information indicating an activity of the subject. The learning data generation unit 314 generates learning data representing a relationship between the stored information indicating the emotion of the subject (preferably obtained by a first obtaining unit, see embodiment 2), and the stored information indicating the activity of the subject (preferably obtained by a second obtaining unit, see embodiment 2) and store the learning data into a memory. The emotion estimation unit (315) estimates, after the learning data is generated, a current emotion of the subject based on information indicating a current activity of the subject obtained by an obtaining unit (the second obtaining unit, see embodiment 2; this is acquired or obtained in correspondence of the estimation), and the learning data stored in the memory. The control unit 390 provides the subject with a healthcare support feedback based on the estimated current emotion.

The feedback may be represented for instance by one or more messages (in the form of text, audio, and/or video, etc.) suggesting certain activities to undertake or lifestyle to follow, or one or more stimuli signals induced on the subject (for instance, audio/video signal to induce stimulation on the subject, and/or an electric signal inducing stimulation on the subject, etc.). For example, the feedback may be provided when the estimated arousal value and/or the estimated valence value meet a predetermined condition; also, the feedback may be provided at a higher frequency (i.e. more frequently) when the estimated arousal value and/or the estimated valence value becomes larger (e.g. the subject with higher arousal value and/or valence value would be more actively following the suggestion message, such that higher effects can be expected by correspondingly applying the feedback), or the content the feedback may be changed depending on whether the estimated arousal value and/or the estimated valence value are positive values or negative values. In this way, the subject can be guided/instructed or physically stimulated towards maintain a good health condition, or improving his/her health condition. Other types of feedback are of course suitable.

Further, the apparatus of the present embodiment may optionally comprise a cognitive state estimating unit configured to determine a cognitive state of the subject based on further information indicating an activity of the subject, wherein the control unit is further configured to provide the subject with a healthcare support feedback further based on the cognitive state. In this way, an even more accurate mental state can be estimated, such that the feedback can be more accurately established (e.g. the feedback is correctly generated when the mental state is accurately estimated, i.e. the feedback is generated when really needed).

Further optionally, the healthcare support feedback comprises any combination amongst healthcare support information (e.g. a message in the form of text, audio, image, and/or video, see above examples also) and health case support stimulus (e.g. an electric stimulus applied to the subject).

Further optionally, the control unit is configured to provide a degree of healthcare support feedback corresponding to at least one amongst a degree of estimated arousal and a degree of estimated valence included in the estimated current emotion (in other words, if at least one of arousal/valence degree is increasing, the degree of support is also increasing; similarly and optionally, in the decreasing case). By degree of healthcare support it is herein meant a level of support that is provided to the subject in order to maintain and/or improve his/her health condition. For instance, a high level of support implies providing feedback more frequently, of having higher impact (e.g. audiovisual feedback having higher impact than simple text feedback) or being more intense (e.g. in the case of a stimulus, a stronger electric stimulus). For degree of arousal and emotion we refer to what has been stated previously. Thus, when for instance the degree of arousal and/or valence decreases, the degree of healthcare support increases, or vice versa. As for other embodiments, the inverse relationship between the support degree and the valence/arousal degree can be of any type (linear, non-linear, etc.).

Optionally, the apparatus of the present embodiment can also be represented like in FIG. 16B, noting that same reference signs indicate same components as in FIG. 2. Also, the storage unit 320, the learning data generation unit 314, and the emotion estimation unit 315 of FIG. 16B correspond to the units 20, 14, and 15, respectively, of FIG. 2. Thus, reference is made to embodiment 2 for further optional details, applicable also to the present embodiment.

An operation (method) of an apparatus for healthcare support of a subject according to the present embodiment is now described with reference to FIG. 16C. At step S3110, information indicating an emotion of a subject, and information indicating an activity of the subject are stored. At step S3113, learning data are generated, representing a relationship between the stored information indicating the emotion of the subject, and the information indicating the activity of the subject obtained by an obtaining unit and store the learning data into a memory. At step S3123, it is estimated, after the learning data is generated, a current emotion of the subject based on stored information indicating a current activity of the subject, and the learning data stored in the memory. At step S3190, the subject is provided with a healthcare support feedback based on the estimated current emotion.

Other operations or method steps are immediately evident from the respective description of the apparatus according to the present embodiment and the above.

### Other Embodiments

This invention is not limited to the above embodiments. For example, the relationship between human emotions, and vital signs and motion information may change depending on the date, the day of the week, the season, the environmental change, and other factors. The learning data may thus be updated regularly or as appropriate. For example, when the difference calculated between a correct value of an emotion and an estimate of the emotion obtained by the emotion estimation unit 15 exceeds a predetermined range of correct values, the learning data stored in the learning data storage 23 is updated. In this case, the correct value can be estimated based on the trends in the emotion estimates. In another embodiment, the correct value of the emotion may be input regularly by the subject through the emotion input device 2.

In the embodiments described above, the information indicating the emotion of the subject is input into the emotion estimation apparatus 1 through the emotion input device 2, which is a smartphone or a tablet terminal. The information may be input in any other manner. For example, the subject may write his or her emotion information on print media such as a questionnaire form, and may use a scanner to read the emotion information and input the information into the emotion estimation apparatus 1.

Further, a camera may be used to detect the facial expression of the subject. The information about the detected facial expression may then be input into the emotion estimation apparatus 1 as emotion information. A microphone may be used to detect the subject's voice. The detection information may then be input into the emotion estimation apparatus 1 as emotion information. Emotion information may be collected from a large number of unspecified individuals by using questionnaires, and the average or other representative values of the collected information may be used as population data to correct the emotion information from an individual. Any other technique may be used to input the information indicating human emotions into the emotion estimation apparatus 1.

The above embodiments describe the two-dimensional arousal-valence system for expressing the information about the subject's emotion. Another method may be used to express the subject's emotion information.

In the embodiments described above, the measurement data items, namely, the heart electrical activity H, the skin potential activity G, the eye movement EM, the motion BM, and the activity amount Ex are input into the emotion estimation apparatus 1 as the information indicating the activity of the subject, and all these items are used to estimate the emotions. However, at least one item of the measurement data may be used to estimate the emotions. For example, the heart electrical activity H, which is highly contributory to emotions among the other vital signs, may be used to estimate the emotions using only the heart electrical activity H as the measurement data. Vital signs other than the items used in the embodiments may also be used.

The emotion estimation apparatus may be a smartphone or a wearable terminal, which may function as the measurement device. The emotion estimation apparatus may also function as the emotion input device.

In addition, the types of vital signs and motion information indicating the activity of a subject, the model representing the relationship between changes in emotions and in vital signs and motion information, the learning data generation procedure and its details, the emotion estimation procedure and its details, and the type and the structure of the emotion estimation apparatus may also be modified variously without departing from the scope and spirit of the invention.

The present invention is not limited to the embodiments described above, but may be embodied using the components modified without departing from the scope and spirit of the invention in its implementation. An appropriate combination of the components described in the embodiments may constitute various aspects of the invention. For example, some of the components described in the embodiments may be eliminated. Further, components from different embodiments may be combined as appropriate.

The above embodiments may be partially or entirely expressed in, but not limited to, the following forms.

### APPENDIX 1

An emotion estimation apparatus that allows information transmission between an emotion input device for receiving an emotion of a subject expressed as arousal and valence information, and a measurement device for measuring a condition of the subject and outputting measurement information, the apparatus comprising a hardware processor and a memory,

the hardware processor being configured to

obtain emotion information that is input through the emotion input device; obtain the measurement information from the measurement device;

generate, in a learning mode, learning data representing a relationship between first emotion information and first measurement information, and store the learning data into the memory, the first emotion information being the obtained emotion information, the first measurement information being the obtained measurement information; and

estimate, in an emotion estimation mode after the learning data is generated, second emotion information corresponding to second measurement information based on the second measurement information that is the obtained measurement information and the learning data stored in the memory.

### APPENDIX 2

An emotion estimation method implemented by an apparatus including at least one hardware processor and a memory, the method comprising:

obtaining, with the at least one hardware processor and the memory, emotion information that is input through an emotion input device for receiving an emotion of a subject expressed as arousal and valence information;

obtaining, with the at least one hardware processor and the memory, measurement information from a measurement device for measuring a condition of the subject as measurement information, with the at least one hardware processor and the memory;

generating, in a learning mode, with the at least one hardware processor and the memory, learning data representing a relationship between first emotion information and first measurement information, and storing the learning data into the memory, the first emotion information being the obtained emotion information, the first measurement information being the obtained measurement information; and

estimating, in an emotion estimation mode after the learning data is generated, with the at least one hardware processor and the memory, second emotion information corresponding to second measurement information based on the second measurement information that is the obtained measurement information and the learning data stored in the memory.

### REFERENCE SIGNS LIST

1 emotion estimation apparatus
2 emotion input device
3 measurement device
4 communication network
10 control unit
11 scale data obtaining controller
12 measurement data obtaining controller
13 feature quantity extraction unit
14 learning data generation unit
15 emotion estimation unit
16 estimation result output unit
20 storage unit
21 scale data storage
22 measurement data storage
23 learning data storage
30 interface unit

1. An apparatus for assisting driving of a vehicle, the apparatus comprising:
a storage device that stores information indicating an emotion of a subject, and information indicating an activity of the subject, wherein the information indicating an emotion of a subject comprises information relating to physiological parameters obtained by at least one first sensor set according to a first configuration, and wherein the information indicating the activity of the subject comprises information relating to physiological parameters obtained by at least one second sensor set according to a second configuration, wherein one or more of: the first and second sensors are different from each other; and the first and second configurations are different from each other;
a processor configured with a program to perform operations comprising:
operation as a learning data generation unit configured to generate learning data representing a relationship between the stored information indicating the emotion of the subject, and the stored information indicating the activity of the subject and to store the learning data into a memory;
operation as an emotion estimation unit configured to estimate, after the learning data is generated, a current emotion of the subject based on information indicating a current activity of the subject obtained by operation as an obtaining unit, and the learning data stored in the memory; and
operation as an assisting unit configured to provide driving assistance of the vehicle based on the estimated current emotion.

2. The apparatus according to claim 1, wherein
the processor is configured with the program to perform operations further comprising operation as a cognitive state estimating unit configured to determine a cognitive state of the subject based on further information indicating an activity of the subject, and
the processor is configured with the program to perform operations such that operation as the assisting unit is further configured to provide driving assistance of the vehicle further based on the cognitive state.

3. The apparatus according to claim 1, wherein the driving assistance comprises any one or any combination of active control measures of the vehicle by the assisting unit during driving, and providing a driver of the vehicle with at least a feedback during driving.

4. The apparatus according to claim 1, wherein
the information indicating the emotion of the subject is expressed in a two-dimensional coordinate system having a first axis representing an emotional arousal and a second axis representing an emotional valence, and the estimated current emotion is output as values corresponding to the emotional arousal and the emotional valence, and
the processor is configured with the program to perform operations such that operation as the assisting unit is further configured to provide a degree of driving assistance inversely corresponding to at least one of: a degree of the emotional arousal; and a degree of the emotional valence.

5. An apparatus for controlling a manufacturing line, the apparatus comprising:
a storage device that stores information indicating an emotion of a subject, and information indicating an activity of the subject;
a processor configured with a program to perform operations comprising:
operation as a learning data generation unit configured to generate learning data representing a relationship between the stored information indicating the emotion of the subject, and the stored information indicating the activity of the subject and store the learning data into a memory;
operation as an emotion estimation unit configured to estimate, after the learning data is generated, a current emotion of the subject based on information indicating a current activity of the subject obtained by an obtaining unit, and the learning data stored in the memory; and
operation as a control unit configured to control the manufacturing line based on the estimated current emotion.

6. The apparatus according to claim 5, wherein
the information indicating the emotion of the subject comprises information relating to physiological parameters obtained by at least one first sensor set according to a first configuration,
the information indicating the activity of the subject comprises information relating to physiological parameters obtained by at least one second sensor set according to a second configuration, and
one or more of: the first and second sensors are different from each other; and the first and second configurations are different from each other.

7. The apparatus according to claim 5, wherein
the processor is configured with the program to perform operations further comprising operation as a cognitive state estimating unit configured to determine a cognitive state of the subject based on further information indicating an activity of the subject, and
the processor is configured with the program to perform operations such that operation as the control unit is further configured to control the manufacturing line further based on the cognitive state.

8. The apparatus according to claim 5, wherein the processor is configured with the program to perform operations such that operation as the control unit is further configured to perform any combination of: controlling a speed of movement of a manufacturing line component, and controlling a speed of operation of a manufacturing line component.

9. The apparatus according to claim 5, wherein
the information indicating an emotion of a subject is expressed in a two-dimensional coordinate system having a first axis representing an emotional arousal and a second axis representing an emotional valence, and the estimated current emotion is output as values corresponding to the emotional arousal and the emotional valence, and
the processor is configured with the program to perform operations such that operation as the control unit is further configured to provide a degree of control of the manufacturing line inversely corresponding to at least one of: a degree of the emotional arousal; and a degree of the emotional valence.

10. An apparatus for healthcare support of a subject, the apparatus comprising:
a storage device that stores information indicating an emotion of a subject, and information indicating an activity of the subject;
a processor configured with a program to perform operations comprising:
operation as a learning data generation unit configured to generate learning data representing a relationship between the stored information indicating the emotion of the subject, and the stored information indicating the activity of the subject and store the learning data into a memory;
operation as an emotion estimation unit configured to estimate, after the learning data is generated, a current emotion of the subject based on information indicating a current activity of the subject obtained by an obtaining unit, and the learning data stored in the memory; and
operation as a control unit configured to provide the subject with a healthcare support feedback based on the estimated current emotion.

11. The apparatus according to claim 10, wherein
the information indicating the emotion of the subject comprises information relating to physiological parameters obtained by at least one first sensor set according to a first configuration,
the information indicating the activity of the subject comprises information relating to physiological parameters obtained by at least one second sensor set according to a second configuration, and
one or more of: the first and second sensors are different from each other; and the first and second configurations are different from each other.

12. The apparatus according to claim 10, wherein
the processor is configured with the program to perform operations further comprising operation as a cognitive state estimating unit configured to determine a cognitive state of the subject based on further information indicating an activity of the subject, and
the processor is configured with the program to perform operations such that operation as the control unit is further configured to provide the subject with a healthcare support feedback further based on the cognitive state.

13. The apparatus according to claim 10, wherein the healthcare support feedback comprises any combination of healthcare support information and health case support stimulus.

14. The apparatus according to claim 10, wherein
the information indicating the emotion of the subject is expressed preferably in a two-dimensional coordinate system having a first axis representing an emotional arousal and a second axis representing an emotional valence,
the estimated current emotion is output as values corresponding to the emotional arousal and the emotional valence, and
the processor is configured with the program perform operations such that operation as the control unit is further configured to provide a degree of healthcare support feedback corresponding to at least one of: a degree of the emotional arousal; and a degree of the emotional valence.

15. An emotion estimation apparatus, comprising a processor configured with a program to perform operations comprising:
operation as a first obtaining unit configured to obtain information indicating an emotion of a subject;
operation as a second obtaining unit configured to obtain information indicating an activity of the subject;
operation as a learning data generation unit configured to generate learning data representing a relationship between the information indicating the emotion of the subject obtained by the first obtaining unit, and the information indicating the activity of the subject obtained by the second obtaining unit and store the learning data into a memory; and
operation as an emotion estimation unit configured to estimate, after the learning data is generated, a current emotion of the subject based on information indicating a current activity of the subject obtained by the second obtaining unit, and the learning data stored in the memory.

16. The apparatus according to claim 15, wherein
the information indicating the emotion of the subject comprises information relating to physiological parameters obtained by at least one first sensor set according to a first configuration,
the information indicating the activity of the subject comprises information relating to physiological parameters obtained by at least one second sensor set according to a second configuration, and
one or more of: the first and second sensors are different from each other; and the first and second configurations are different from each other.

17. The emotion estimation apparatus according to claim 15, wherein
the processor is configured with the program to perform operations such that operation as the learning data generation unit is further configured to generate a regression equation representing a relationship between the information indicating the emotion of the subject and the information indicating the activity with a correct value being the information indicating the emotion of the subject obtained by the first obtaining unit, and a variable being the information indicating the activity of the subject concurrently obtained by the second obtaining unit, and to store the generated regression equation into the memory as the learning data.

18. The emotion estimation apparatus according to claim 15, wherein the processor is configured with the program to perform operations such that:
operation as the first obtaining unit is further configured to obtain information about emotional arousal and emotional valence as information indicating the emotion of the subject, and
operation as the learning data generation unit is further configured to generate a regression equation representing a relationship between the information indicating the emotion of the subject and the information indicating the activity for the emotional arousal and for the emotional valence, and to store each generated regression equation into the memory as the learning data.

19. The emotion estimation apparatus according to claim 15, wherein
the processor is configured with the program to perform operations such that operation as the learning data generation unit is further configured to define a plurality of windows each having a predetermined unit duration and being arranged at time points chronologically shifted from one another, and to generate, for each window, learning data representing a relationship between a change in the information indicating the emotion of the subject and a change in the information indicating the activity of the subject in each window.

20. The emotion estimation apparatus according to claim 19, wherein the processor is configured with the program to perform operations such that operation as the learning data generation unit comprises:
operation as a generator configured to generate, for every change of a predetermined value in at least one of the unit duration of the window or the chronological shift of the window, learning data representing a relationship between a change in the information indicating the emotion of the subject and a change in the information indicating the activity of the subject in the window; and
operation as a selector configured to calculate, for each generated learning data set, a difference between a change in information about an estimate of the emotion obtained based on the learning data and a change in information about a correct value of the emotion obtained by the first obtaining unit, and select at least one of the unit duration or the chronological shift of the window that minimizes the difference.

21. The emotion estimation apparatus according to claim 15, wherein
the processor is configured with the program to perform operations such that operation as the second obtaining unit is further configured to obtain measurement information comprising a measurement result of at least one of heart electrical activity, skin potential activity, eye movement, motion, or an activity amount as information indicating the activity of the subject.

22. The emotion estimation apparatus according to claim 15, wherein the processor is configured with the program to perform operations further comprising:
operation as a learning data updating unit configured to compare an emotion value estimated by the emotion estimation unit with a range of correct values for the emotion, and update the learning data stored in the memory based on a result of the comparison.

23. An emotion estimation method implemented by an emotion estimation apparatus including a processor and a memory, the method comprising:
obtaining information indicating an emotion of a subject;
obtaining information indicating an activity of the subject;
generating learning data representing a relationship between the obtained information indicating the emotion of the subject and the obtained information indicating the activity of the subject, and storing the learning data into the memory; and
obtaining, after the learning data is generated, information indicating a current activity of the subject, and estimating a current emotion of the subject based on the obtained information indicating the current activity and the learning data stored in the memory.

24. The method according to claim 23, wherein
the information indicating an emotion of a subject comprises information relating to physiological parameters obtained by at least one first sensor set according to a first configuration,
the information indicating the activity of the subject comprises information relating to physiological parameters obtained by at least one second sensor set according to a second configuration, and
one or more of: the first and second sensors are different from each other; and the first and second configurations are different from each other.

25. A non-transitory computer-readable storage medium storing an emotion estimation program, which when read and executed, causes a processor to function as each component included in the emotion estimation apparatus according to claim 15.

26. A method for assisting driving of a vehicle, the method comprising:
storing information indicating an emotion of a subject, and information indicating an activity of the subject, wherein the information indicating an emotion of a subject comprises information relating to physiological parameters obtained by at least one first sensor set according to a first configuration, and wherein the information indicating the activity of the subject comprises information relating to physiological parameters obtained by at least one second sensor set according to a second configuration, wherein one or more of: the first and second sensors are different from each other; and the first and second configurations are different from each other;
generating learning data representing a relationship between the stored information indicating the emotion of the subject, and the stored information indicating the activity of the subject and store the learning data into a memory;
estimating, after the learning data is generated, a current emotion of the subject based on information indicating a current activity of the subject obtained by an obtaining unit, and the learning data stored in the memory; and
providing driving assistance of the vehicle based on the estimated current emotion.

27. A method for controlling a manufacturing line, the method comprising:
storing information indicating an emotion of a subject, and information indicating an activity of the subject;
generating learning data representing a relationship between the stored information indicating the emotion of the subject, and the stored information indicating the activity of the subject and store the learning data into a memory;
estimating, after the learning data is generated, a current emotion of the subject based on information indicating a current activity of the subject obtained by an obtaining unit, and the learning data stored in the memory; and
controlling the manufacturing line based on the estimated current emotion.

28. The method according to claim 27, wherein the information indicating an emotion of a subject comprises information relating to physiological parameters obtained by at least one first sensor set according to a first configuration, and wherein the information indicating the activity of the subject comprises information relating to physiological parameters obtained by at least one second sensor set according to a second configuration, wherein one or more of: the first and second sensors are different from each other; and the first and second configurations are different from each other.

29. A method for healthcare support of a subject, the method comprising:
storing information indicating an emotion of a subject, and information indicating an activity of the subject;
generating learning data representing a relationship between the stored information indicating the emotion of the subject, and the information indicating the activity of the subject obtained by an obtaining unit and store the learning data into a memory;
estimating, after the learning data is generated, a current emotion of the subject based on stored information indicating a current activity of the subject, and the learning data stored in the memory; and
providing the subject with a healthcare support feedback based on the estimated current emotion.

30. The method according to claim 29, wherein
the information indicating an emotion of a subject comprises information relating to physiological parameters obtained by at least one first sensor set according to a first configuration,
the information indicating the activity of the subject comprises information relating to physiological parameters obtained by at least one second sensor set according to a second configuration, and
one of: the first and second sensors are different from each other; and the first and second configurations are different from each other.

31. A non-transitory computer-readable storage medium storing a computer program, which when read and executed, causes a computer to perform the steps according to claim 26.

32. The apparatus according to claim 1, wherein the information indicating the emotion of the subject is expressed in a two-dimensional coordinate system having a first axis representing an arousal state and a second axis representing a valence state, and the estimated current emotion is output as values corresponding to the arousal state and the valence state.

33. The method according to claim 26, wherein the information indicating the emotion of the subject is expressed in a two-dimensional coordinate system having a first axis representing an arousal state and a second axis representing a valence state, and the estimated current emotion is output as values corresponding to the arousal state and the valence state.

34. The apparatus according to claim 19, wherein the predetermined unit duration of a window is determined on the basis of at least one of:
obtained activity information indicating an activity state of the subject; and
an interaction time interval indicating a time length for an interacting operation between a device coupled to the apparatus and the subject.

35. The apparatus according to claim 34, wherein the device coupled to the apparatus comprises one of: a vehicle; a component of a manufacturing line; and a healthcare feedback providing device, and wherein, respectively, the interaction time interval comprises a time length for an interacting operation between the subject and the vehicle, a time length for an interacting operation between the subject and the component of the manufacturing line, and a time length for an interacting operation between the subject and the feedback providing device.
